(19) United States
(12) Patent Application Publication
(10) Pub. No.: US 2008/0113962 A1
(43) Pub. Date: May 15, 2008

(54) Condensed Tricyclic Benzimidazoles For the Treatment of Gastrointestinal Disorders

(75) Inventors: Peter Jan Zimmermann, Radolfzell (DE); Wilm Buhr, Konstanz (DE); Christof Brehm, Konstanz (DE); Andreas Palmer, Singen (DE); M. Vittoria Chiesa, Konstanz (DE); Wolfgang-Alexander Simon, Konstanz (DE); Stefan Postius, Konstanz (DE); Wolfgang Kromer, Konstanz (DE)

Correspondence Address: NATH & ASSOCIATES PLLC, 112 South West Street, Alexandria, VA, 22314, US

(73) Assignee: Aitana Pharma AG, Konstanz (DE)

(21) Appl. No.: 11/663,920

(22) PCT Filed: Sep. 30, 2005
(86) PCT No.: PCT/EP05/54944

§ 371 Date: May 22, 2007

(30) Foreign Application Priority Data
Oct. 4, 2004 (EP) 04104851.3

(51) Int. Cl. A61K 31/5377; A61K 31/496; A61K 31/4745; A61K 31/454; A61K 31/4188; C07D 491/02; C07D 471/02

(52) U.S. Cl. 514/210.21; 514/233.2; 514/253.03; 514/254.06; 514/292; 514/393; 544/126; 544/361; 546/84; 514/322; 546/199; 548/302.1

(57) ABSTRACT

The invention relates to condensed tricyclic benzimidazoles of formula (I)

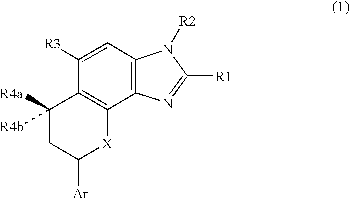

in which the substituents and symbols have the meanings indicated in the description. The compounds have gastric secretion inhibiting and excellent gastric and intestinal protective action properties.

### TECHNICAL FIELD

The invention relates to novel compounds, which are used in the pharmaceutical industry as active compounds for the production of medicaments.

### BACKGROUND ART

In European patent application 266326 (which corresponds to U.S. Pat. No. 5,106,862), benzimidazole derivatives having a very broad variety of substituents are disclosed, which are said to be active as anti-ulcer agents. In J. Med. Chem. 1991, 34, 533-541 (Kaminski et al.), the inhibition of gastric H⁺/K⁺-ATPase by certain substituted imidazo[1,2-a]pyridines is described. Kaminski et al. describe in a later publication (J. Med. Chem. 1997, 40, 427-436) the results of a detailed analysis of the same and similar imidazo[1,2-a]pyridines. Tricyclic imidazo[1,2-a]pyridines with a specific substitution pattern are described in the International Patent Application WO 95/27714 (Astra AB). In the International Patent Application WO 03/014123 (ALTANA Pharma AG), new imidazo[1,2-a]pyridines with a certain substitution pattern are disclosed. The compounds described therein are unsubstituted in 7- and 8-position. In the International Patent Application WO 97/47603 (Astra AB) benzimidazoles with a specific benzyloxy or benzylamino substitution are described. In the International Patent Application WO 04/054984 (ALTANA Pharma AG), benzimidazole derivatives with a variety of substituents are disclosed, which are said to be active as anti-ulcer agents.

The International Patent Application WO 04/087701 (ALTANA Pharma AG) discloses substituted, tricyclic benzimidazole derivatives, which are unsubstituted in 6- and 7-position, which compounds have gastric secretion inhibiting and excellent gastric and intestinal protective action properties.

The International Patent Application WO 05/058893 (ALTANA Pharma AG) discloses substituted, tricyclic benzimidazole derivatives, which are substituted in 6- and 7-position, which compounds likewise have gastric secretion inhibiting and excellent gastric and intestinal protective action properties.

### SUMMARY OF THE INVENTION

The invention relates to condensed tricyclic benzimidazoles of the formula 1

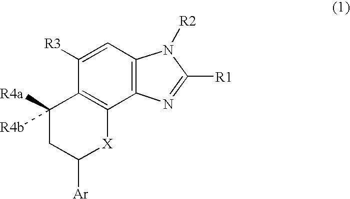

in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl, mono- or di-1-4C-alkylamino or 1-4C-alkylcarbonyloxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, mono- or di-1-4C-alkylamino 1-4C-alkylcarbonyl, hydroxy-1-4C-alkyl, fluoro-2-4C-alkyl, aryl-1-4C-alkoxy-1-4C-alkyl, hydroxy or 1-4C-alkoxy,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy-1-4C-alkoxy, 1-4C-alkylcarbonylamino, 1-4C-alkylcarbonyl-N-1-4C-alkylamino, 1-4C-alkoxy-1-4C-alkylcarbonylamino or the group —CO—NR31R32, where
   R31 is hydrogen, hydroxy, 1-7C-alkyl, 3-7C-cycloalkyl, hydroxy-1-4C-alkyl or 1-4C-alkoxy-1-4C-alkyl and
   R32 is hydrogen, 1-7C-alkyl, 3-7C-cycloalkyl, hydroxy-1-4C-alkyl or 1-4C-alkoxy-1-4C-alkyl, or where
   R31 and R32 together, including the nitrogen atom to which both are bonded, are a pyrrolidino, hydroxy-pyrrolidino, aziridino, azetidino, piperidino, piperazino, N-1-4C-alkylpiperazino or morpholino group,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl or 1-4C-alkoxy-1-4C-alkyl,
X is O (oxygen) or NH and
Ar is a mono- or bicyclic aromatic residue, substituted by R5, R6, R7 and R8, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   wherein
   R5 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R6 is hydrogen, 1-4C-alkyl, 1-4C-alkoxy, 1-4C-alkoxycarbonyl, halogen, trifluoromethyl or hydroxy,
   R7 is hydrogen, 1-4C-alkyl or halogen and
   R8 is hydrogen, 1-4C-alkyl or halogen,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

1-4C-Alkyl represents a straight-chain or branched alkyl group having 1 to 4 carbon atoms. Examples which may be mentioned are the butyl, isobutyl, sec-butyl, tert-butyl, propyl, isopropyl, ethyl and the methyl group.

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkyl-1-4C-alkyl represents one of the aforementioned 1-4C-alkyl groups, which is substituted by one of the aforementioned 3-7C-cycloalkyl groups. Examples which may be mentioned are the cyclopropylmethyl, the cyclohexylmethyl and the cyclohexylethyl group.

1-4C-Alkoxy represents a group, which in addition to the oxygen atom contains one of the aforementioned 1-4C-alkyl groups. Examples which may be mentioned are the butoxy, isobutoxy, sec-butoxy, tert-butoxy, propoxy, isopropoxy and preferably the ethoxy and methoxy group.

1-4C-Alkoxy-1-4C-alkyl represents one of the aforementioned 1-4C-alkyl groups, which is substituted by one of the aforementioned 1-4C-alkoxy groups. Examples which may be mentioned are the methoxymethyl, the methoxyethyl group and the butoxyethyl group.

1-4C-Alkoxycarbonyl (1-4C-alkoxy-CO—) represents a carbonyl group, to which one of the aforementioned 1-4C-alkoxy groups is bonded. Examples which may be mentioned are the methoxycarbonyl (CH₃O—C(O)—) and the ethoxycarbonyl group (CH₃CH₂O—C(O)—).

2-4C-Alkenyl represents a straight-chain or branched alkenyl group having 2 to 4 carbon atoms. Examples which may be mentioned are the 2-butenyl, 3-butenyl, 1-propenyl and the 2-propenyl group (allyl group).

2-4C-Alkynyl represents a straight-chain or branched alkynyl group having 2 to 4 carbon atoms. Examples which may be mentioned are the 2-butynyl, 3-butynyl, and preferably the 2-propynyl, group (propargyl group).

Fluoro-1-4C-alkyl represents one of the aforementioned 1-4C-alkyl groups, which is substituted by one or more fluorine atoms. An example which may be mentioned are the trifluoromethyl group, the difluoromethyl, the 2-fluoroethyl, the 2,2-difluoroethyl or the 2,2,2-trifluoroethyl group.

Hydroxy-1-4C-alkyl represents one of the aforementioned 1-4C-alkyl groups, which is substituted by a hydroxy group. Examples which may be mentioned are the hydroxymethyl, the 2-hydroxyethyl and the 3-hydroxypropyl group. Hydroxy-1-4C-alkyl within the scope of the invention is understood to include 1-4C-alkyl groups with two or more hydroxy groups. Examples which may be mentioned are the 3,4-dihydroxybutyl and in particular the 2,3-dihydroxypropyl group.

Mono- or di-1-4C-alkylamino represents an amino group, which is substituted by one or by two identical or different—groups from the aforementioned 1-4C-alkyl groups. Examples which may be mentioned are the dimethylamino, the diethylamino and the diisopropylamino group.

1-4C-Alkylcarbonyloxy represents a 1-4C-alkylcarbonyl group which is bonded to an oxygen atom. An example which may be mentioned is the acetoxy group (CH₃CO—O—).

1-4C-Alkylcarbonyloxy-1-4C-alkyl represents one of the aforementioned 1-4C-alkyl groups, which is substituted by one of the aforementioned 1-4C-alkylcarbonyloxy groups. An example which may be mentioned is the acetoxymethyl group (CH₃CO—O—CH₂).

Mono- or di-1-4C-alkylamino-1-4C-alkylcarbonyl represents a 1-4C-alkylcarbonyl group, which is substituted by a mono- or di-1-4C-alkylamino groups. Examples, which may be mentioned, are the dimethylamino-methylcarbonyl and the dimethylamino-ethylcarbonyl group.

Fluoro-2-4C-alkyl represents a 2-4C-alkyl group, which is substituted by one or more fluorine atoms. Examples which may be mentioned are the 2-fluoroethyl, the 2,2-difluoroethyl or the 2,2,2-trifluoroethyl groups.

Aryl-1-4C-alkoxy represents one of the aforementioned 1-4C-alkoxy groups, which is substituted by one of the abovementioned aryl groups. An exemplary preferred aryl-1-4C-alkoxy group is the benzyloxy group.

Aryl-1-4C-alkoxy-1-4C-alkyl denotes one of the aforementioned 1-4C-alkyl groups, which is substituted by one of the aforementioned aryl-1-4C-alkoxy radicals. An example which may be mentioned is the benzyloxymethyl radical.

Halogen within the meaning of the invention is bromo, chloro and fluoro.

1-4C-Alkoxy-1-4C-alkoxy represents one of the aforementioned 1-4C-alkoxy groups, which is substituted by a further 1-4C-alkoxy group. Examples which may be mentioned are the groups 2-(methoxy)ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxy-1-4C-alkyl represents one of the aforementioned 1-4C-alkoxy-1-4C-alkyl groups, which is substituted by one of the aforementioned 1-4C-alkoxy groups. An example which may be mentioned is the group 2-(methoxy)ethoxymethyl (CH₃—O—CH₂—CH₂—O—CH₂—).

Fluoro-1-4C-alkoxy represents one of the aforementioned 1-4C-alkoxy groups, which is completely or mainly substituted by fluorine, “mainly” meaning in this connection that more than half of the hydrogen atoms are replaced by fluorine atoms. Examples of completely or mainly fluoro-substituted 1-4C-alkoxy groups which may be mentioned are the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy group

Fluoro-1-4C-alkoxy-1-4C-alkyl represents one of the aforementioned 1-4C-alkyl groups, which is substituted by a fluoro-1-4C-alkoxy group. Examples of fluoro-1-4C-alkoxy-1-4C-alkyl groups are the 1,1,2,2-tetrafluoroethoxymethyl, the 2,2,2-trifluoroethoxymethyl, the trifluoromethoxyethyl and the difluoromethoxyethyl group.

1-4C-Alkylcarbonylamino represents an amino group to which a 1-4C-alkylcarbonyl group is bonded. Examples which may be mentioned are the propionylamino (C3H7C(O)NH—) and the acetylamino group (acetamido group) (CH3C(O)NH—).

1-4C-Alkylcarbonyl-N-1-4C-alkylamino represents an 1-4C-alkylamino group to which a 1-4C-alkylcarbonyl group is bonded. Examples which may be mentioned are the propionyl-N-methylamino (C3H7C(O)NCH3-) and the acetyl-N-methylamino group (CH3C(O)NCH3-).

1-4C-Alkoxy-1-4C-alkylcarbonylamino represents a 1-4C-alkylcarbonylamino represents an amino group to which a 1-4C-alkoxy group is bonded. Examples which may be mentioned are the methoxypropionylamino (CH₃O—C₃H₆C(O)NH—) and the methoxy-acetylamino group (CH₃O—CH₂C(O)NH—).

1-7C-Alkyl represents a straight-chain or branched alkyl group having 1 to 7 carbon atoms. Examples which may be mentioned are the heptyl, isoheptyl (5-methylhexyl), hexyl, isohexyl (4-methylpentyl), neohexyl (3,3-dimethylbutyl), pentyl, isopentyl (3-methylbutyl), neopentyl (2,2-dimethylpropyl), butyl, isobutyl, sec-butyl, tert-butyl, propyl, isopropyl, ethyl and the methyl group.

Groups Ar which may be mentioned are, for example, the following substituents: 4-acetoxyphenyl, 4-acetamidophenyl, 2-methoxyphenyl, 3-methoxyphenyl, 4-methoxyphenyl, 3-benzyloxyphenyl, 4-benzyloxyphenyl, 3-benzyloxy-4-methoxyphenyl, 4-benzyloxy-3-methoxyphenyl, 3,5-bis(trifluoromethyl)phenyl, 4-butoxyphenyl, 2-chlorophenyl, 3-chlorophenyl, 4-chlorophenyl, 2-chloro-6-fluorophenyl, 3-chloro-4-fluorophenyl, 2-chloro-5-nitrophenyl, 4-chloro-3-nitrophenyl, 3-(4-chlorophenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)-3-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-trifluoromethoxyphenyl)-2-pyrrolyl, 1-(2-nitrobenzyl)-2-pyrrolyl, 1-(4-ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorphenoxy)-4-pyrazolyl, 1-methyl-3-trifluomethyl-5-(3-trifluoromethylphenoxy)-4-pyrazolyl, 4-methoxycarbonyl-1-(2,6-dichlorophenyl)-5-pyrazolyl, 5-allyloxy-1-methyl-3-trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)-2-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxycarbonyl-6-methyl-4-phenyl-3-pyridyl, 2-chloro-3-pyridyl, 6-(3-trifluoromethylphenoxy)-3-pyridyl, 2-(4-chlorophenoxy)-3-pyridyl, 2,4-dimethoxy-5-pyrimidinyl, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl.

2-4C-Alkenyloxy represents a group, which in addition to the oxygen atom contains one of the above-mentioned 2-4C-alkenyl groups. Examples, which may be mentioned, are the 2-butenyloxy, 3-butenyloxy, 1-propenyloxy and the 2-propenyloxy group (allyloxy group).

1-4C-Alkylcarbonyl represents a group, which in addition to the carbonyl group contains one of the abovementioned 1-4C-alkyl groups. An example which may be mentioned is the acetyl group. Carboxy-1-4C-alkyl represents a 1-4C-alkyl group which is substituted by a carboxyl group. Examples, which may be mentioned, are the carboxymethyl and the 2-carboxyethyl group.

1-4C-Alkoxycarbonyl-1-4C-alkyl represents a 1-4C-alkyl group, which is substituted by one of the abovementioned 1-4C-alkoxycarbonyl groups. Examples, which may be mentioned, are the Methoxycarbonylmethyl and the ethoxycarbonylmethyl group.

Aryl-1-4C-alkyl represents one of the aforementioned 1-4C-alkyl groups, which is substituted by one of the abovementioned aryl groups. An exemplary preferred aryl-1-4C-alkyl group is the benzyl group.

1-4C-Alkoxycarbonylamino represents an amino group, which is substituted by one of the aforementioned 1-4C-alkoxycarbonyl groups. Examples, which may be mentioned, are the ethoxycarbonylamino and the methoxycarbonylamino group.

1-4C-Alkoxy-1-4C-alkoxycarbonyl represents a carbonyl group, to which one of the aforementioned 1-4C-alkoxy-1-4C-alkoxy groups is bonded. Examples which may be mentioned are the 2-(methoxy)ethoxycarbonyl (CH₃—O—CH₂CH₂—O—CO—) and the 2-(ethoxy)ethoxycarbonyl group (CH₃CH₂—O—CH₂CH₂—O—CO—).

1-4C-Alkoxy-1-4C-alkoxycarbonylamino represents an amino group, which is substituted by one of the aforementioned 1-4C-alkoxy-1-4C-alkoxycarbonyl groups. Examples which may be mentioned are the 2-(methoxy)ethoxycarbonylamino and the 2-(ethoxy)ethoxycarbonylamino group.

Possible salts of compounds of the formula 1—depending on substitution—are especially all acid addition salts. Particular mention may be made of the pharmacologically tolerable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are used in salt preparation—depending on whether a mono- or polybasic acid is concerned and on which salt is desired—in an equimolar quantitative ratio or one differing therefrom.

Pharmacologically intolerable salts, which can initially be obtained, for example, as process products in the production of the compounds according to the invention on the industrial scale, are converted into the pharmacologically tolerable salts by processes known to the person skilled in the art.

It is known to the person skilled in the art that the compounds according to the invention and their salts, if, for example, they are isolated in crystalline form, can contain various amounts of solvents. The invention therefore also comprises all solvates and in particular all hydrates of the compounds of the formula 1, and also all solvates and in particular all hydrates of the salts of the compounds of the formula 1.

The compounds of the formula 1 have chirality centers in the 6- and 8-positions. The invention thus relates to all enantiomers (diastereomers) in any desired mixing ratio to another, including the pure enantiomers, which are a preferred subject of the invention.

One aspect of the invention are compounds of formula 1, in which R3 is 1-4C-alkylcarbonyl-N-1-4C-alkylamino and R1, R2, R4a, R4b, X and Ar have the meanings given above, and the salts of these compounds.

Another aspect of the invention are compounds of formula 1, in which R3 is the group —CO—NR31R32 in which R31 and R32 together, including the nitrogen atom to which both are bonded, are a hydroxypyrrolidino group and R1, R2, R4a, R4b, X and Ar have the meanings given above, and the salts of these compounds.

Yet another aspect of the invention are compounds of formula 1, in which R3 is the group —CO—NR31R32 in which R31 and R32 together, including the nitrogen atom to which both are bonded, are an aziridino group and R1, R2, R4a, R4b, X and Ar have the meanings given above, and the salts of these compounds.

Yet another aspect of the invention are compounds of formula 1, in which R3 is the group —CO—NR31R32 in which R31 and R32 together, including the nitrogen atom to which both are bonded, are an azetidino group and R1, R2, R4a, R4b, X and Ar have the meanings given above, and the salts of these compounds.

Yet another aspect of the invention are compounds of formula 1, in which one of R4a and R4b is hydrogen and the other is hydroxy and R1, R2, X and Ar have the meanings given above, and the salts of these compounds.

Yet another aspect of the invention are compounds of formula 1, in which one of R4a and R4b is hydrogen and the other is 1-4C-alkoxy and R1, R2, X and Ar have the meanings given above, and the salts of these compounds.

Yet another aspect of the invention are compounds of formula 1, in which one of R4a and R4b is hydrogen and the other is 1-4C-alkoxy-1-4C-alkoxy and R1, R2, X and Ar have the meanings given above, and the salts of these compounds.

Yet another aspect of the invention are compounds of formula 1, in which one of R4a and R4b is hydroxy and the other is 1-4C-alkyl and R1, R2, X and Ar have the meanings given above, and the salts of these compounds.

One embodiment (embodiment a) of the invention are compounds of formula 1, in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 24C-alkenyl, 2-4C-alkynyl, fluoro-1-4C-alkyl, hydroxy-1-4C-alkyl, mono- or di-1-4C-alkylamino or 1-4C-alkylcarbonyloxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, mono- or di-1-4C-alkylamino 1-4C-alkylcarbonyl, hydroxy-1-4C-alkyl, fluoro-2-4C-alkyl, aryl-1-4C-alkoxy-1-4C-alkyl, hydroxy or 1-4C-alkoxy,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxy, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, piperidino, piperazino, N-1-4C-alkylpiperazino or morpholino group,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl or 1-4C-alkoxy-1-4C-alkyl,
X is O (oxygen) or NH and
Ar is a mono- or bicyclic aromatic residue, substituted by R5, R6, R7 and R8, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   wherein
   R5 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R6 is hydrogen, 1-4C-alkyl, 1-4C-alkoxy, 1-4C-alkoxycarbonyl, halogen, trifluoromethyl or hydroxy,
   R7 is hydrogen, 1-4C-alkyl or halogen and
   R8 is hydrogen, 1-4C-alkyl or halogen,
   and wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano,
      and the salts of these compounds.

Another embodiment (embodiment b) of the invention are compounds of the formula 1, in which X is O (oxygen) and R1, R2, R3, R4a, R4b and Ar have the meanings given above, and the salts of these compounds.

Another embodiment (embodiment c) of the invention are compounds of the formula 1, in which X is NH and R1, R2, R3, R4a, R4b and Ar have the meanings given above, and the salts of these compounds.

Another embodiment (embodiment d) of the invention are compounds of the formula 1, in which one of R4a and R4b is hydrogen and the other is hydroxy, 1-4C-alkoxy or 1-4C-alkoxy-1-4C-alkoxy and R1, R2, R3, X and Ar have the meanings given above, and the salts of these compounds.

Another embodiment (embodiment e) of the invention are compounds of the formula 1, in which one of R4a and R4b is hydroxy and the other is 1-4C-alkyl or 1-4C-alkoxy-1-4C-alkyl and R1, R2, R3, X and Ar have the meanings given above, and the salts of these compounds.

Compounds to be mentioned particularly are those of formula 1,

in which
R1 is hydrogen, 1-4C-alkyl, 3-7C-cycloalkyl or fluoro-1-4C-alkyl,
R2 is hydrogen or 1-4C-alkyl,
R3 is hydrogen, halogen, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl or the group —CO—NR31R32,
   where
   R31 is hydrogen, 1-4C-alkyl, 3-7C-cycloalkyl, hydroxy-1-4C-alkyl or 1-4C-alkoxy-1-4C-alkyl and
   R32 is hydrogen or 1-4C-alkyl,
   or where
   R31 and R32 together, including the nitrogen atom to which both are bonded, are a pyrrolidino, hydroxypyrrolidino, aziridino, azetidino, piperidino, piperazino, N-1-4C-alkylpiperazino or morpholino group,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl or 1-4C-alkoxy-1-4C-alkyl,
X is O (oxygen) or NH and
Ar is a phenyl group, substituted by R5, R6, R7 and R8,
   wherein
   R5 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R6 is hydrogen, 1-4C-alkyl, 1-4C-alkoxy, 1-4C-alkoxycarbonyl, halogen, trifluoromethyl or hydroxy,
   R7 is hydrogen, 1-4C-alkyl or halogen and
   R8 is hydrogen, 1-4C-alkyl or halogen,
      and the salts of these compounds.

Among the compounds of formula 1, those of the formula 1a are preferred

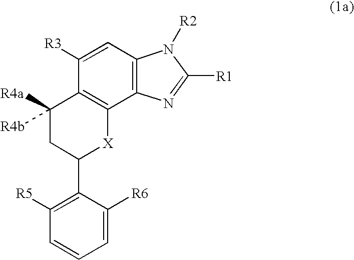

in which
R1 is hydrogen, 1-4C-alkyl, 3-7C-cycloalkyl or fluoro-1-4C-alkyl,
R2 is hydrogen or 1-4C-alkyl,
R3 is hydrogen, halogen, carboxyl, 1-4C-alkoxycarbonyl, hydroxy-1-4C-alkyl, 1-4C-alkoxy-1-4C-alkyl, 1-4C-alkoxy-1-4C-alkoxy-1 AC-alkyl or the group —CO—NR31R32,
   where
   R31 is hydrogen, 1-4C-alkyl, 3-7C-cycloalkyl, hydroxy-1-4C-alkyl or 1-4C-alkoxy-1-4C-alkyl and
   R32 is hydrogen or 1-4C-alkyl,
   or where
   R31 and R32 together, including the nitrogen atom to which both are bonded, are a pyrrolidino, hydroxypyrrolidino, aziridino, azetidino, piperidino, piperazino, N-1-4C-alkylpiperazino or morpholino group,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl,
R5 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R6 is hydrogen, 1-4C-alkyl or 1-4C-alkoxy and
X is O (oxygen) or NH,
   and the salts of these compounds.

Among the compounds of formula 1, those of the formula 1a are particularly preferred

in which
R1 is 1-4C-alkyl, 3-7C-cycloalkyl or fluoro-1-4C-alkyl,
R2 is hydrogen or 1-4C-alkyl,
R3 is hydrogen, carboxyl, hydroxy-1-4C-alkyl, 1-4C-alkoxy-1-4C-alkyl, 1-4C-alkoxy-1-4C-alkoxy-1-4C-alkyl or the group —CO—NR31R32,
   where
   R31 is hydrogen, 1-4C-alkyl, hydroxy-1-4C-alkyl or 1-4C-alkoxy-1-4C-alkyl and
   R32 is hydrogen or 1-4C-alkyl, or where
   R31 and R32 together, including the nitrogen atom to which both are bonded, are a pyrrolidino, aziridino, azetidino or morpholino group,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl,
R5 is hydrogen,
R6 is hydrogen and
X is O (oxygen) or NH,
   and the salts of these compounds.

Exemplified compounds to be mentioned particularly are those of formula 1a,

in which
R1 is 1-4C-alkyl,
R2 is hydrogen or 1-4C-alkyl,
R3 is hydrogen or the group —CO—NR31R32,
   where
   R31 is hydrogen or 1-4C-alkyl and
   R32 is 1-4C-alkyl, one of R4a and R4b is hydrogen and the other is hydroxy, 1-4C-alkoxy or 1-4C-alkoxy-1-4C-alkoxy, or
one of R4a and R4b is hydroxy and the other is 1-4C-alkyl,
R5 is hydrogen,
R6 is hydrogen and
X is O (oxygen) or NH,
   and the salts of these compounds.

Exemplified compounds also to be mentioned particularly are those of formula 1a,

in which
R1 is 1-4C-alkyl,
R2 is hydrogen or 1-4C-alkyl,
R3 is hydrogen or the group —CO—NR31R32,
   where
   R31 is 1-4C-alkyl and
   R32 is 1-4C-alkyl,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl,
R5 is hydrogen,
R6 is hydrogen and
X is O (oxygen) or NH,
   and the salts of these compounds.

Emphasis is given to compounds of the formula 1a, in which
R1 is 1-4C-alkyl,
R2 is 1-4C-alkyl,
R3 is hydrogen or the group —CO—NR31R32,
   where
   R31 is 1-4C-alkyl and
   R32 is 1-4C-alkyl,
one of R4a and R4b is hydrogen and the other is hydroxy, 1-4C-alkoxy or 1-4C-alkoxy-1-4C-alkoxy,
R5 is hydrogen,
R6 is hydrogen and
X is O (oxygen) or NH,
   and the salts of these compounds.

Preferred subject of the invention are compounds of the formula 2

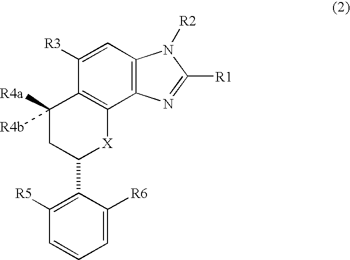

in which
R1 is 1-4C-alkyl or 3-7C-cycloalkyl,
R2 is hydrogen or 1-4C-alkyl,
R3 is 1-4C-alkoxycarbonyl, hydroxy-1-4C-alkyl, 1-4C-alkoxy-1-4C-alkyl or the group —CO—NR31R32,
   where
   R31 is hydrogen, 1-4C-alkyl, hydroxy-1-4C-alkyl or 1-4C-alkoxy-1-4C-alkyl and
   R32 is hydrogen or 1-4C-alkyl,
   or where
   R31 and R32 together, including the nitrogen atom to which both are bonded, are a pyrrolidino, aziridino, azetidino or morpholino group,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl or 1-4C-alkoxy-1-4C-alkyl,
R5 is hydrogen,
R6 is hydrogen and
X is O (oxygen) or NH,
   and the salts of these compounds.

Preferred exemplified compounds of the invention are those compounds of the formula 2,

in which
R1 is 1-4C-alkyl,
R2 is hydrogen or 1-4C-alkyl,
R3 is hydrogen or the group —CO—NR31R32,
   where
   R31 is hydrogen or 1-4C-alkyl and
   R32 is 1-4C-alkyl,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl,
R5 is hydrogen,
R6 is hydrogen and
X is O (oxygen) or NH,
   and the salts of these compounds.

Preferred exemplified compounds of the invention are also those compounds of the formula 2,
in which
R1 is 1-4C-alkyl,
R2 is hydrogen or 1-4C-alkyl,
R3 is hydrogen or the group —CO—NR31R32,
   where
   R31 is 1-4C-alkyl and
   R32 is 1-4C-alkyl,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl,
R5 is hydrogen,
R6 is hydrogen and
X is O (oxygen) or NH,
   and the salts of these compounds.

Emphasis is given to those compounds of the formula 2
R1 is 1-4C-alkyl,
R2 is 1-4C-alkyl,
R3 is hydrogen or the group —CO—NR31R32,
   where
   R31 is 1-4C-alkyl and
   R32 is 1-4C-alkyl,
one of R4a and R4b is hydrogen and the other is hydroxy, 1-4C-alkoxy or 1-4C-alkoxy-1-4C-alkoxy,
R5 is hydrogen,
R6 is hydrogen and
X is O (oxygen) or NH,
   and the salts of these compounds.

Exemplary compounds are those of the formula 1a, in which R1, R2, R3, R4a, R4b and X have the meanings given in the following table 1 (Me=CH₃, Et=C₂H₅) and R5 and R6 are hydrogen, and the salts of these compounds.

Exemplary preferred compounds are those of the formula 2, in which R1, R2, R3, R4a, R4b and X have the meanings given in the following table 1 (Me=CH₃, Et=C₂H₅) and R5 and R6 are hydrogen, and the salts of these compounds.

TABLE 1

| R1 | R2 | R3 | R4a | R4b | X |
|---|---|---|---|---|---|
| Me | Me | CH₂OH | OH | H | O |
| Me | Me | CH₂OH | H | OH | O |
| Me | Me | CH₂OH | OMe | H | O |
| Me | Me | CH₂OH | H | OMe | O |
| Me | Me | CH₂OH | OEt | H | O |
| Me | Me | CH₂OH | H | OEt | O |
| Me | Me | CH₂OH | OCH₂CH₂OMe | H | O |
| Me | Me | CH₂OH | H | OCH₂CH₂OMe | O |
| Me | Me | CH₂OCH₃ | OH | H | O |
| Me | Me | CH₂OCH₃ | H | OH | O |
| Me | Me | CH₂OCH₃ | OMe | H | O |
| Me | Me | CH₂OCH₃ | H | OMe | O |
| Me | Me | CH₂OCH₃ | OEt | H | O |
| Me | Me | CH₂OCH₃ | H | OEt | O |
| Me | Me | CH₂OCH₃ | OCH₂CH₂OMe | H | O |
| Me | Me | CH₂OCH₃ | H | OCH₂CH₂OMe | O |
| Me | Me | CONHMe | OH | H | O |
| Me | Me | CONHMe | H | OH | O |
| Me | Me | CONHMe | OMe | H | O |
| Me | Me | CONHMe | H | OMe | O |
| Me | Me | CONHMe | OEt | H | O |
| Me | Me | CONHMe | H | OEt | O |
| Me | Me | CONHMe | OCH₂CH₂OMe | H | O |
| Me | Me | CONHMe | H | OCH₂CH₂OMe | O |
| Me | Me | CONHMe | O-n-butyl | H | O |
| Me | Me | CONHMe | H | O-n-butyl | O |
| Me | Me | CON-pyrrolidine | OH | H | O |
| Me | Me | CON-pyrrolidine | H | OH | O |
| Me | Me | CON-pyrrolidine | OMe | H | O |
| Me | Me | CON-pyrrolidine | H | OMe | O |
| Me | Me | CON-pyrrolidine | OEt | H | O |
| Me | Me | CON-pyrrolidine | H | OEt | O |
| Me | Me | CON-pyrrolidine | OCH₂CH₂OMe | H | O |
| Me | Me | CON-pyrrolidine | H | OCH₂CH₂OMe | O |
| Me | Me | CONH(CH₂)₂OH | OH | H | O |
| Me | Me | CONH(CH₂)₂OH | H | OH | O |
| Me | Me | CONH(CH₂)₂OH | OMe | H | O |
| Me | Me | CONH(CH₂)₂OH | H | OMe | O |
| Me | Me | CONH(CH₂)₂OH | OEt | H | O |
| Me | Me | CONH(CH₂)₂OH | H | OEt | O |
| Me | Me | CONH(CH₂)₂OH | OCH₂CH₂OMe | H | O |
| Me | Me | CONH(CH₂)₂OH | H | OCH₂CH₂OMe | O |
| Me | Me | CONH(CH₂)₂OMe | OH | H | O |
| Me | Me | CONH(CH₂)₂OMe | H | OH | O |
| Me | Me | CONH(CH₂)₂OMe | OMe | H | O |
| Me | Me | CONH(CH₂)₂OMe | H | OMe | O |
| Me | Me | CONH(CH₂)₂OMe | OEt | H | O |
| Me | Me | CONH(CH₂)₂OMe | H | OEt | O |
| Me | Me | CONH(CH₂)₂OMe | OCH₂CH₂OMe | H | O |
| Me | Me | CONH(CH₂)₂OMe | H | OCH₂CH₂OMe | O |
| Me | Me | CONH₂ | OH | H | O |
| Me | Me | CONH₂ | H | OH | O |
| Me | Me | CONH₂ | OMe | H | O |
| Me | Me | CONH₂ | H | OMe | O |
| Me | Me | CONH₂ | OEt | H | O |
| Me | Me | CONH₂ | H | OEt | O |
| Me | Me | CONH₂ | OCH₂CH₂OMe | H | O |
| Me | Me | CONH₂ | H | OCH₂CH₂OMe | O |
| Me | Me | CON-morpholine | OH | H | O |
| Me | Me | CON-morpholine | H | OH | O |
| Me | Me | CON-morpholine | OMe | H | O |
| Me | Me | CON-morpholine | H | OMe | O |
| Me | Me | CON-morpholine | OEt | H | O |
| Me | Me | CON-morpholine | H | OEt | O |
| Me | Me | CON-morpholine | OCH₂CH₂OMe | H | O |
| Me | Me | CON-morpholine | H | OCH₂CH₂OMe | O |
| Me | Me | CONMe₂ | OH | H | O |
| Me | Me | CONMe₂ | H | OH | O |
| Me | Me | CONMe₂ | OMe | H | O |
| Me | Me | CONMe₂ | H | OMe | O |
| Me | Me | CONMe₂ | OEt | H | O |
| Me | Me | CONMe₂ | H | OEt | O |
| Me | Me | CONMe₂ | OCH₂CH₂OMe | H | O |
| Me | Me | CONMe₂ | H | OCH₂CH₂OMe | O |
| Me | Me | CONMe₂ | O-n-butyl | H | O |
| Me | Me | CONMe₂ | H | O-n-butyl | O |
| Me | Me | CH₂O(CH₂)₂OMe | OH | H | O |
| Me | Me | CH₂O(CH₂)₂OMe | H | OH | O |
| Me | Me | CH₂O(CH₂)₂OMe | OMe | H | O |
| Me | Me | CH₂O(CH₂)₂OMe | H | OMe | O |
| Me | Me | CH₂O(CH₂)₂OMe | OEt | H | O |
| Me | Me | CH₂O(CH₂)₂OMe | H | OEt | O |
| Me | Me | CH₂O(CH₂)₂OMe | OCH₂CH₂OMe | H | O |
| Me | Me | CH₂O(CH₂)₂OMe | H | OCH₂CH₂OMe | O |
| Me | Me | CON-aziridine | OH | H | O |
| Me | Me | CON-aziridine | H | OH | O |
| Me | Me | CON-aziridine | OMe | H | O |
| Me | Me | CON-aziridine | H | OMe | O |
| Me | Me | CON-aziridine | OEt | H | O |
| Me | Me | CON-aziridine | H | OEt | O |
| Me | Me | CON-aziridine | OCH₂CH₂OMe | H | O |
| Me | Me | CON-aziridine | H | OCH₂CH₂OMe | O |
| Me | Me | COOEt | OH | H | O |
| Me | Me | COOEt | H | OH | O |
| Me | Me | COOEt | OMe | H | O |
| Me | Me | COOEt | H | OMe | O |
| Me | Me | COOEt | OEt | H | O |
| Me | Me | COOEt | H | OEt | O |
| Me | Me | COOEt | OCH₂CH₂OMe | H | O |
| Me | Me | COOEt | H | OCH₂CH₂OMe | O |
| Me | Me | COOH | OH | H | O |
| Me | Me | COOH | H | OH | O |
| Me | Me | COOH | OMe | H | O |
| Me | Me | COOH | H | OMe | O |
| Me | Me | COOH | OEt | H | O |
| Me | Me | COOH | H | OEt | O |
| Me | Me | COOH | OCH₂CH₂OMe | H | O |
| Me | Me | COOH | H | OCH₂CH₂OMe | O |
| Me | Me | CON-azetidine | OH | H | O |
| Me | Me | CON-azetidine | H | OH | O |
| Me | Me | CON-azetidine | OMe | H | O |
| Me | Me | CON-azetidine | H | OMe | O |
| Me | Me | CON-azetidine | OEt | H | O |
| Me | Me | CON-azetidine | H | OEt | O |
| Me | Me | CON-azetidine | OCH₂CH₂OMe | H | O |
| Me | Me | CON-azetidine | H | OCH₂CH₂OMe | O |
| Me | Me | CONH(CH₂)₂Me | OH | H | O |
| Me | Me | CONH(CH₂)₂Me | H | OH | O |
| Me | Me | CONH(CH₂)₂Me | OMe | H | O |
| Me | Me | CONH(CH₂)₂Me | H | OMe | O |
| Me | Me | CONH(CH₂)₂Me | OEt | H | O |
| Me | Me | CONH(CH₂)₂Me | H | OEt | O |
| Me | Me | CONH(CH₂)₂Me | OCH₂CH₂OMe | H | O |
| Me | Me | CONH(CH₂)₂Me | H | OCH₂CH₂OMe | O |
| Me | Me | CONHCH₂CHOHCH₂OH | OH | H | O |
| Me | Me | CONHCH₂CHOHCH₂OH | H | OH | O |
| Me | Me | CONHCH₂CHOHCH₂OH | OMe | H | O |
| Me | Me | CONHCH₂CHOHCH₂OH | H | OMe | O |
| Me | Me | CONHCH₂CHOHCH₂OH | OEt | H | O |
| Me | Me | CONHCH₂CHOHCH₂OH | H | OEt | O |
| Me | Me | CONHCH₂CHOHCH₂OH | OCH₂CH₂OMe | H | O |
| Me | Me | CONHCH₂CHOHCH₂OH | H | OCH₂CH₂OMe | O |
| Me | Me | NCH₃COCH₃ | OH | H | O |
| Me | Me | NCH₃COCH₃ | H | OH | O |
| Me | Me | NCH₃COCH₃ | OMe | H | O |
| Me | Me | NCH₃COCH₃ | H | OMe | O |
| Me | Me | NCH₃COCH₃ | OEt | H | O |
| Me | Me | NCH₃COCH₃ | H | OEt | O |
| Me | Me | NCH₃COCH₃ | OCH₂CH₂OMe | H | O |
| Me | Me | NCH₃COCH₃ | H | OCH₂CH₂OMe | O |
| Me | Me | NHCOCH₃ | OH | H | O |
| Me | Me | NHCOCH₃ | H | OH | O |
| Me | Me | NHCOCH₃ | OMe | H | O |
| Me | Me | NHCOCH₃ | H | OMe | O |
| Me | Me | NHCOCH₃ | OEt | H | O |
| Me | Me | NHCOCH₃ | H | OEt | O |
| Me | Me | NHCOCH₃ | OCH₂CH₂OMe | H | O |
| Me | Me | NHCOCH₃ | H | OCH₂CH₂OMe | O |
| Me | Me | NHCOCH₂OMe | OH | H | O |
| Me | Me | NHCOCH₂OMe | H | OH | O |
| Me | Me | NHCOCH₂OMe | OMe | H | O |
| Me | Me | NHCOCH₂OMe | H | OMe | O |
| Me | Me | NHCOCH₂OMe | OEt | H | O |
| Me | Me | NHCOCH₂OMe | H | OEt | O |
| Me | Me | NHCOCH₂OMe | OCH₂CH₂OMe | H | O |
| Me | Me | NHCOCH₂OMe | H | OCH₂CH₂OMe | O |
| Me | Me | NHCO(CH₂)₂OMe | OH | H | O |
| Me | Me | NHCO(CH₂)₂OMe | H | OH | O |
| Me | Me | NHCO(CH₂)₂OMe | OMe | H | O |
| Me | Me | NHCO(CH₂)₂OMe | H | OMe | O |
| Me | Me | NHCO(CH₂)₂OMe | OEt | H | O |
| Me | Me | NHCO(CH₂)₂OMe | H | OEt | O |
| Me | Me | NHCO(CH₂)₂OMe | OCH₂CH₂OMe | H | O |
| Me | Me | NHCO(CH₂)₂OMe | H | OCH₂CH₂OMe | O |
| Me | Me | OCH₂OMe | OH | H | O |
| Me | Me | OCH₂OMe | H | OH | O |
| Me | Me | OCH₂OMe | OMe | H | O |
| Me | Me | OCH₂OMe | H | OMe | O |
| Me | Me | OCH₂OMe | OEt | H | O |
| Me | Me | OCH₂OMe | H | OEt | O |
| Me | Me | OCH₂OMe | OCH₂CH₂OMe | H | O |
| Me | Me | OCH₂OMe | H | OCH₂CH₂OMe | O |
| Me | Me | O(CH₂)₂OMe | OH | H | O |
| Me | Me | O(CH₂)₂OMe | H | OH | O |
| Me | Me | O(CH₂)₂OMe | OMe | H | O |
| Me | Me | O(CH₂)₂OMe | H | OMe | O |
| Me | Me | O(CH₂)₂OMe | OEt | H | O |
| Me | Me | O(CH₂)₂OMe | H | OEt | O |
| Me | Me | O(CH₂)₂OMe | OCH₂CH₂OMe | H | O |
| Me | Me | O(CH₂)₂OMe | H | OCH₂CH₂OMe | O |
| Me | Me | CONH-cyclopropyl | OH | H | O |
| Me | Me | CONH-cyclopropyl | H | OH | O |
| Me | Me | CONH-cyclopropyl | OMe | H | O |
| Me | Me | CONH-cyclopropyl | H | OMe | O |
| Me | Me | CONH-cyclopropyl | OEt | H | O |
| Me | Me | CONH-cyclopropyl | H | OEt | O |
| Me | Me | CONH-cyclopropyl | OCH₂CH₂OMe | H | O |
| Me | Me | CONH-cyclopropyl | H | OCH₂CH₂OMe | O |
| Me | Me | H | OH | H | O |
| Me | Me | H | H | OH | O |
| Me | Me | H | OMe | H | O |
| Me | Me | H | H | OMe | O |
| Me | Me | H | OEt | H | O |
| Me | Me | H | H | OEt | O |
| Me | Me | H | OCH₂CH₂OMe | H | O |
| Me | Me | H | H | OCH₂CH₂OMe | O |
| Me | H | CH₂OH | OH | H | O |
| Me | H | CH₂OH | H | OH | O |
| Me | H | CH₂OH | OMe | H | O |
| Me | H | CH₂OH | H | OMe | O |
| Me | H | CH₂OH | OEt | H | O |
| Me | H | CH₂OH | H | OEt | O |
| Me | H | CH₂OH | OCH₂CH₂OMe | H | O |
| Me | H | CH₂OH | H | OCH₂CH₂OMe | O |
| Me | H | CH₂OCH₃ | OH | H | O |
| Me | H | CH₂OCH₃ | H | OH | O |
| Me | H | CH₂OCH₃ | OMe | H | O |
| Me | H | CH₂OCH₃ | H | OMe | O |
| Me | H | CH₂OCH₃ | OEt | H | O |
| Me | H | CH₂OCH₃ | H | OEt | O |
| Me | H | CH₂OCH₃ | OCH₂CH₂OMe | H | O |
| Me | H | CH₂OCH₃ | H | OCH₂CH₂OMe | O |
| Me | H | CONHMe | OH | H | O |
| Me | H | CONHMe | H | OH | O |
| Me | H | CONHMe | OMe | H | O |
| Me | H | CONHMe | H | OMe | O |
| Me | H | CONHMe | OEt | H | O |
| Me | H | CONHMe | H | OEt | O |
| Me | H | CONHMe | OCH₂CH₂OMe | H | O |
| Me | H | CONHMe | H | OCH₂CH₂OMe | O |
| Me | H | CON-pyrrolidine | OH | H | O |
| Me | H | CON-pyrrolidine | H | OH | O |
| Me | H | CON-pyrrolidine | OMe | H | O |
| Me | H | CON-pyrrolidine | H | OMe | O |
| Me | H | CON-pyrrolidine | OEt | H | O |
| Me | H | CON-pyrrolidine | H | OEt | O |
| Me | H | CON-pyrrolidine | OCH₂CH₂OMe | H | O |
| Me | H | CON-pyrrolidine | H | OCH₂CH₂OMe | O |
| Me | H | CONH(CH₂)₂OH | OH | H | O |
| Me | H | CONH(CH₂)₂OH | H | OH | O |
| Me | H | CONH(CH₂)₂OH | OMe | H | O |
| Me | H | CONH(CH₂)₂OH | H | OMe | O |
| Me | H | CONH(CH₂)₂OH | OEt | H | O |
| Me | H | CONH(CH₂)₂OH | H | OEt | O |
| Me | H | CONH(CH₂)₂OH | OCH₂CH₂OMe | H | O |
| Me | H | CONH(CH₂)₂OH | H | OCH₂CH₂OMe | O |
| Me | H | CONH(CH₂)₂OMe | OH | H | O |
| Me | H | CONH(CH₂)₂OMe | H | OH | O |
| Me | H | CONH(CH₂)₂OMe | OMe | H | O |
| Me | H | CONH(CH₂)₂OMe | H | OMe | O |
| Me | H | CONH(CH₂)₂OMe | OEt | H | O |
| Me | H | CONH(CH₂)₂OMe | H | OEt | O |
| Me | H | CONH(CH₂)₂OMe | OCH₂CH₂OMe | H | O |
| Me | H | CONH(CH₂)₂OMe | H | OCH₂CH₂OMe | O |
| Me | H | CONH₂ | OH | H | O |
| Me | H | CONH₂ | H | OH | O |
| Me | H | CONH₂ | OMe | H | O |
| Me | H | CONH₂ | H | OMe | O |
| Me | H | CONH₂ | OEt | H | O |
| Me | H | CONH₂ | H | OEt | O |
| Me | H | CONH₂ | OCH₂CH₂OMe | H | O |
| Me | H | CONH₂ | H | OCH₂CH₂OMe | O |
| Me | H | CON-morpholine | OH | H | O |
| Me | H | CON-morpholine | H | OH | O |
| Me | H | CON-morpholine | OMe | H | O |
| Me | H | CON-morpholine | H | OMe | O |
| Me | H | CON-morpholine | OEt | H | O |
| Me | H | CON-morpholine | H | OEt | O |
| Me | H | CON-morpholine | OCH₂CH₂OMe | H | O |
| Me | H | CON-morpholine | H | OCH₂CH₂OMe | O |
| Me | H | CONMe₂ | OH | H | O |
| Me | H | CONMe₂ | H | OH | O |
| Me | H | CONMe₂ | OMe | H | O |
| Me | H | CONMe₂ | H | OMe | O |
| Me | H | CONMe₂ | OEt | H | O |
| Me | H | CONMe₂ | H | OEt | O |
| Me | H | CONMe₂ | OCH₂CH₂OMe | H | O |
| Me | H | CONMe₂ | H | OCH₂CH₂OMe | O |
| Me | H | CH₂O(CH₂)₂OMe | OH | H | O |
| Me | H | CH₂O(CH₂)₂OMe | H | OH | O |
| Me | H | CH₂O(CH₂)₂OMe | OMe | H | O |
| Me | H | CH₂O(CH₂)₂OMe | H | OMe | O |
| Me | H | CH₂O(CH₂)₂OMe | OEt | H | O |
| Me | H | CH₂O(CH₂)₂OMe | H | OEt | O |
| Me | H | CH₂O(CH₂)₂OMe | OCH₂CH₂OMe | H | O |
| Me | H | CH₂O(CH₂)₂OMe | H | OCH₂CH₂OMe | O |
| Me | H | CON-aziridine | OH | H | O |
| Me | H | CON-aziridine | H | OH | O |
| Me | H | CON-aziridine | OMe | H | O |
| Me | H | CON-aziridine | H | OMe | O |
| Me | H | CON-aziridine | OEt | H | O |
| Me | H | CON-aziridine | H | OEt | O |
| Me | H | CON-aziridine | OCH₂CH₂OMe | H | O |
| Me | H | CON-aziridine | H | OCH₂CH₂OMe | O |
| Me | H | COOEt | OH | H | O |
| Me | H | COOEt | H | OH | O |
| Me | H | COOEt | OMe | H | O |
| Me | H | COOEt | H | OMe | O |
| Me | H | COOEt | OEt | H | O |
| Me | H | COOEt | H | OEt | O |
| Me | H | COOEt | OCH₂CH₂OMe | H | O |
| Me | H | COOEt | H | OCH₂CH₂OMe | O |
| Me | H | COOH | OH | H | O |
| Me | H | COOH | H | OH | O |
| Me | H | COOH | OMe | H | O |
| Me | H | COOH | H | OMe | O |
| Me | H | COOH | OEt | H | O |
| Me | H | COOH | H | OEt | O |
| Me | H | COOH | OCH₂CH₂OMe | H | O |
| Me | H | COOH | H | OCH₂CH₂OMe | O |
| Me | H | CON-azetidine | OH | H | O |
| Me | H | CON-azetidine | H | OH | O |
| Me | H | CON-azetidine | OMe | H | O |
| Me | H | CON-azetidine | H | OMe | O |
| Me | H | CON-azetidine | OEt | H | O |
| Me | H | CON-azetidine | H | OEt | O |
| Me | H | CON-azetidine | OCH₂CH₂OMe | H | O |
| Me | H | CON-azetidine | H | OCH₂CH₂OMe | O |
| Me | H | CONH(CH₂)₂Me | OH | H | O |
| Me | H | CONH(CH₂)₂Me | H | OH | O |
| Me | H | CONH(CH₂)₂Me | OMe | H | O |
| Me | H | CONH(CH₂)₂Me | H | OMe | O |
| Me | H | CONH(CH₂)₂Me | OEt | H | O |
| Me | H | CONH(CH₂)₂Me | H | OEt | O |
| Me | H | CONH(CH₂)₂Me | OCH₂CH₂OMe | H | O |
| Me | H | CONH(CH₂)₂Me | H | OCH₂CH₂OMe | O |
| Me | H | CONHCH₂CHOHCH₂OH | OH | H | O |
| Me | H | CONHCH₂CHOHCH₂OH | H | OH | O |
| Me | H | CONHCH₂CHOHCH₂OH | OMe | H | O |
| Me | H | CONHCH₂CHOHCH₂OH | H | OMe | O |
| Me | H | CONHCH₂CHOHCH₂OH | OEt | H | O |
| Me | H | CONHCH₂CHOHCH₂OH | H | OEt | O |
| Me | H | CONHCH₂CHOHCH₂OH | OCH₂CH₂OMe | H | O |
| Me | H | CONHCH₂CHOHCH₂OH | H | OCH₂CH₂OMe | O |
| Me | H | NCH₃COCH₃ | OH | H | O |
| Me | H | NCH₃COCH₃ | H | OH | O |
| Me | H | NCH₃COCH₃ | OMe | H | O |
| Me | H | NCH₃COCH₃ | H | OMe | O |
| Me | H | NCH₃COCH₃ | OEt | H | O |
| Me | H | NCH₃COCH₃ | H | OEt | O |
| Me | H | NCH₃COCH₃ | OCH₂CH₂OMe | H | O |
| Me | H | NCH₃COCH₃ | H | OCH₂CH₂OMe | O |
| Me | H | NHCOCH₃ | OH | H | O |
| Me | H | NHCOCH₃ | H | OH | O |
| Me | H | NHCOCH₃ | OMe | H | O |
| Me | H | NHCOCH₃ | H | OMe | O |
| Me | H | NHCOCH₃ | OEt | H | O |
| Me | H | NHCOCH₃ | H | OEt | O |
| Me | H | NHCOCH₃ | OCH₂CH₂OMe | H | O |
| Me | H | NHCOCH₃ | H | OCH₂CH₂OMe | O |
| Me | H | NHCOCH₂OMe | OH | H | O |
| Me | H | NHCOCH₂OMe | H | OH | O |
| Me | H | NHCOCH₂OMe | OMe | H | O |
| Me | H | NHCOCH₂OMe | H | OMe | O |
| Me | H | NHCOCH₂OMe | OEt | H | O |
| Me | H | NHCOCH₂OMe | H | OEt | O |
| Me | H | NHCOCH₂OMe | OCH₂CH₂OMe | H | O |
| Me | H | NHCOCH₂OMe | H | OCH₂CH₂OMe | O |
| Me | H | NHCO(CH₂)₂OMe | OH | H | O |
| Me | H | NHCO(CH₂)₂OMe | H | OH | O |
| Me | H | NHCO(CH₂)₂OMe | OMe | H | O |
| Me | H | NHCO(CH₂)₂OMe | H | OMe | O |
| Me | H | NHCO(CH₂)₂OMe | OEt | H | O |
| Me | H | NHCO(CH₂)₂OMe | H | OEt | O |
| Me | H | NHCO(CH₂)₂OMe | OCH₂CH₂OMe | H | O |
| Me | H | NHCO(CH₂)₂OMe | H | OCH₂CH₂OMe | O |
| Me | H | OCH₂OMe | OH | H | O |
| Me | H | OCH₂OMe | H | OH | O |
| Me | H | OCH₂OMe | OMe | H | O |
| Me | H | OCH₂OMe | H | OMe | O |
| Me | H | OCH₂OMe | OEt | H | O |
| Me | H | OCH₂OMe | H | OEt | O |
| Me | H | OCH₂OMe | OCH₂CH₂OMe | H | O |
| Me | H | OCH₂OMe | H | OCH₂CH₂OMe | O |
| Me | H | O(CH₂)₂OMe | OH | H | O |
| Me | H | O(CH₂)₂OMe | H | OH | O |
| Me | H | O(CH₂)₂OMe | OMe | H | O |
| Me | H | O(CH₂)₂OMe | H | OMe | O |
| Me | H | O(CH₂)₂OMe | OEt | H | O |
| Me | H | O(CH₂)₂OMe | H | OEt | O |
| Me | H | O(CH₂)₂OMe | OCH₂CH₂OMe | H | O |
| Me | H | O(CH₂)₂OMe | H | OCH₂CH₂OMe | O |
| Me | H | CONH-cyclopropyl | OH | H | O |
| Me | H | CONH-cyclopropyl | H | OH | O |
| Me | H | CONH-cyclopropyl | OMe | H | O |
| Me | H | CONH-cyclopropyl | H | OMe | O |
| Me | H | CONH-cyclopropyl | OEt | H | O |
| Me | H | CONH-cyclopropyl | H | OEt | O |
| Me | H | CONH-cyclopropyl | OCH₂CH₂OMe | H | O |
| Me | H | CONH-cyclopropyl | H | OCH₂CH₂OMe | O |
| Me | H | H | OH | H | O |
| Me | H | H | H | OH | O |
| Me | H | H | OMe | H | O |
| Me | H | H | H | OMe | O |
| Me | H | H | OEt | H | O |
| Me | H | H | H | OEt | O |
| Me | H | H | OCH₂CH₂OMe | H | O |
| Me | H | H | H | OCH₂CH₂OMe | O |
| Me | Me | CH₂OH | OH | H | NH |
| Me | Me | CH₂OH | H | OH | NH |
| Me | Me | CH₂OH | OMe | H | NH |
| Me | Me | CH₂OH | H | OMe | NH |
| Me | Me | CH₂OH | OEt | H | NH |
| Me | Me | CH₂OH | H | OEt | NH |
| Me | Me | CH₂OH | OCH₂CH₂OMe | H | NH |
| Me | Me | CH₂OH | H | OCH₂CH₂OMe | NH |
| Me | Me | CH₂OCH₃ | OH | H | NH |
| Me | Me | CH₂OCH₃ | H | OH | NH |
| Me | Me | CH₂OCH₃ | OMe | H | NH |
| Me | Me | CH₂OCH₃ | H | OMe | NH |
| Me | Me | CH₂OCH₃ | OEt | H | NH |
| Me | Me | CH₂OCH₃ | H | OEt | NH |
| Me | Me | CH₂OCH₃ | OCH₂CH₂OMe | H | NH |
| Me | Me | CH₂OCH₃ | H | OCH₂CH₂OMe | NH |
| Me | Me | CONHMe | OH | H | NH |
| Me | Me | CONHMe | H | OH | NH |
| Me | Me | CONHMe | OMe | H | NH |
| Me | Me | CONHMe | H | OMe | NH |
| Me | Me | CONHMe | OEt | H | NH |
| Me | Me | CONHMe | H | OEt | NH |
| Me | Me | CONHMe | OCH₂CH₂OMe | H | NH |
| Me | Me | CONHMe | H | OCH₂CH₂OMe | NH |
| Me | Me | CONHMe | O-n-butyl | H | NH |
| Me | Me | CONHMe | H | O-n-butyl | NH |
| Me | Me | CON-pyrrolidine | OH | H | NH |
| Me | Me | CON-pyrrolidine | H | OH | NH |
| Me | Me | CON-pyrrolidine | OMe | H | NH |
| Me | Me | CON-pyrrolidine | H | OMe | NH |
| Me | Me | CON-pyrrolidine | OEt | H | NH |
| Me | Me | CON-pyrrolidine | H | OEt | NH |
| Me | Me | CON-pyrrolidine | OCH₂CH₂OMe | H | NH |
| Me | Me | CON-pyrrolidine | H | OCH₂CH₂OMe | NH |
| Me | Me | CONH(CH₂)₂OH | OH | H | NH |
| Me | Me | CONH(CH₂)₂OH | H | OH | NH |
| Me | Me | CONH(CH₂)₂OH | OMe | H | NH |
| Me | Me | CONH(CH₂)₂OH | H | OMe | NH |
| Me | Me | CONH(CH₂)₂OH | OEt | H | NH |
| Me | Me | CONH(CH₂)₂OH | H | OEt | NH |
| Me | Me | CONH(CH₂)₂OH | OCH₂CH₂OMe | H | NH |
| Me | Me | CONH(CH₂)₂OH | H | OCH₂CH₂OMe | NH |
| Me | Me | CONH(CH₂)₂OMe | OH | H | NH |
| Me | Me | CONH(CH₂)₂OMe | H | OH | NH |
| Me | Me | CONH(CH₂)₂OMe | OMe | H | NH |
| Me | Me | CONH(CH₂)₂OMe | H | OMe | NH |
| Me | Me | CONH(CH₂)₂OMe | OEt | H | NH |
| Me | Me | CONH(CH₂)₂OMe | H | OEt | NH |
| Me | Me | CONH(CH₂)₂OMe | OCH₂CH₂OMe | H | NH |
| Me | Me | CONH(CH₂)₂OMe | H | OCH₂CH₂OMe | NH |
| Me | Me | CONH₂ | OH | H | NH |
| Me | Me | CONH₂ | H | OH | NH |
| Me | Me | CONH₂ | OMe | H | NH |
| Me | Me | CONH₂ | H | OMe | NH |
| Me | Me | CONH₂ | OEt | H | NH |
| Me | Me | CONH₂ | H | OEt | NH |
| Me | Me | CONH₂ | OCH₂CH₂OMe | H | NH |
| Me | Me | CONH₂ | H | OCH₂CH₂OMe | NH |
| Me | Me | CON-morpholine | OH | H | NH |
| Me | Me | CON-morpholine | H | OH | NH |
| Me | Me | CON-morpholine | OMe | H | NH |
| Me | Me | CON-morpholine | H | OMe | NH |
| Me | Me | CON-morpholine | OEt | H | NH |
| Me | Me | CON-morpholine | H | OEt | NH |
| Me | Me | CON-morpholine | OCH₂CH₂OMe | H | NH |
| Me | Me | CON-morpholine | H | OCH₂CH₂OMe | NH |
| Me | Me | CONMe₂ | OH | H | NH |
| Me | Me | CONMe₂ | H | OH | NH |
| Me | Me | CONMe₂ | OMe | H | NH |
| Me | Me | CONMe₂ | H | OMe | NH |
| Me | Me | CONMe₂ | OEt | H | NH |
| Me | Me | CONMe₂ | H | OEt | NH |
| Me | Me | CONMe₂ | OCH₂CH₂OMe | H | NH |
| Me | Me | CONMe₂ | H | OCH₂CH₂OMe | NH |
| Me | Me | CONMe₂ | O-n-butyl | H | NH |
| Me | Me | CONMe₂ | H | O-n-butyl | NH |
| Me | Me | CH₂O(CH₂)₂OMe | OH | H | NH |
| Me | Me | CH₂O(CH₂)₂OMe | H | OH | NH |
| Me | Me | CH₂O(CH₂)₂OMe | OMe | H | NH |
| Me | Me | CH₂O(CH₂)₂OMe | H | OMe | NH |
| Me | Me | CH₂O(CH₂)₂OMe | OEt | H | NH |
| Me | Me | CH₂O(CH₂)₂OMe | H | OEt | NH |
| Me | Me | CH₂O(CH₂)₂OMe | OCH₂CH₂OMe | H | NH |
| Me | Me | CH₂O(CH₂)₂OMe | H | OCH₂CH₂OMe | NH |
| Me | Me | CON-aziridine | OH | H | NH |
| Me | Me | CON-aziridine | H | OH | NH |
| Me | Me | CON-aziridine | OMe | H | NH |
| Me | Me | CON-aziridine | H | OMe | NH |
| Me | Me | CON-aziridine | OEt | H | NH |
| Me | Me | CON-aziridine | H | OEt | NH |
| Me | Me | CON-aziridine | OCH₂CH₂OMe | H | NH |
| Me | Me | CON-aziridine | H | OCH₂CH₂OMe | NH |
| Me | Me | COOEt | OH | H | NH |
| Me | Me | COOEt | H | OH | NH |
| Me | Me | COOEt | OMe | H | NH |
| Me | Me | COOEt | H | OMe | NH |
| Me | Me | COOEt | OEt | H | NH |
| Me | Me | COOEt | H | OEt | NH |
| Me | Me | COOEt | OCH₂CH₂OMe | H | NH |
| Me | Me | COOEt | H | OCH₂CH₂OMe | NH |
| Me | Me | COOH | OH | H | NH |
| Me | Me | COOH | H | OH | NH |
| Me | Me | COOH | OMe | H | NH |
| Me | Me | COOH | H | OMe | NH |
| Me | Me | COOH | OEt | H | NH |
| Me | Me | COOH | H | OEt | NH |
| Me | Me | COOH | OCH₂CH₂OMe | H | NH |
| Me | Me | COOH | H | OCH₂CH₂OMe | NH |
| Me | Me | CON-azetidine | OH | H | NH |
| Me | Me | CON-azetidine | H | OH | NH |
| Me | Me | CON-azetidine | OMe | H | NH |
| Me | Me | CON-azetidine | H | OMe | NH |
| Me | Me | CON-azetidine | OEt | H | NH |
| Me | Me | CON-azetidine | H | OEt | NH |
| Me | Me | CON-azetidine | OCH₂CH₂OMe | H | NH |
| Me | Me | CON-azetidine | H | OCH₂CH₂OMe | NH |
| Me | Me | CONH(CH₂)₂Me | OH | H | NH |
| Me | Me | CONH(CH₂)₂Me | H | OH | NH |
| Me | Me | CONH(CH₂)₂Me | OMe | H | NH |
| Me | Me | CONH(CH₂)₂Me | H | OMe | NH |
| Me | Me | CONH(CH₂)₂Me | OEt | H | NH |
| Me | Me | CONH(CH₂)₂Me | H | OEt | NH |
| Me | Me | CONH(CH₂)₂Me | OCH₂CH₂OMe | H | NH |
| Me | Me | CONH(CH₂)₂Me | H | OCH₂CH₂OMe | NH |
| Me | Me | CONHCH₂CHOHCH₂OH | OH | H | NH |
| Me | Me | CONHCH₂CHOHCH₂OH | H | OH | NH |
| Me | Me | CONHCH₂CHOHCH₂OH | OMe | H | NH |
| Me | Me | CONHCH₂CHOHCH₂OH | H | OMe | NH |
| Me | Me | CONHCH₂CHOHCH₂OH | OEt | H | NH |
| Me | Me | CONHCH₂CHOHCH₂OH | H | OEt | NH |
| Me | Me | CONHCH₂CHOHCH₂OH | OCH₂CH₂OMe | H | NH |
| Me | Me | CONHCH₂CHOHCH₂OH | H | OCH₂CH₂OMe | NH |
| Me | Me | NCH₃COCH₃ | OH | H | NH |
| Me | Me | NCH₃COCH₃ | H | OH | NH |
| Me | Me | NCH₃COCH₃ | OMe | H | NH |
| Me | Me | NCH₃COCH₃ | H | OMe | NH |
| Me | Me | NCH₃COCH₃ | OEt | H | NH |
| Me | Me | NCH₃COCH₃ | H | OEt | NH |
| Me | Me | NCH₃COCH₃ | OCH₂CH₂OMe | H | NH |
| Me | Me | NCH₃COCH₃ | H | OCH₂CH₂OMe | NH |
| Me | Me | NHCOCH₃ | OH | H | NH |
| Me | Me | NHCOCH₃ | H | OH | NH |
| Me | Me | NHCOCH₃ | OMe | H | NH |
| Me | Me | NHCOCH₃ | H | OMe | NH |
| Me | Me | NHCOCH₃ | OEt | H | NH |
| Me | Me | NHCOCH₃ | H | OEt | NH |
| Me | Me | NHCOCH₃ | OCH₂CH₂OMe | H | NH |
| Me | Me | NHCOCH₃ | H | OCH₂CH₂OMe | NH |
| Me | Me | NHCOCH₂OMe | OH | H | NH |
| Me | Me | NHCOCH₂OMe | H | OH | NH |
| Me | Me | NHCOCH₂OMe | OMe | H | NH |
| Me | Me | NHCOCH₂OMe | H | OMe | NH |
| Me | Me | NHCOCH₂OMe | OEt | H | NH |
| Me | Me | NHCOCH₂OMe | H | OEt | NH |
| Me | Me | NHCOCH₂OMe | OCH₂CH₂OMe | H | NH |
| Me | Me | NHCOCH₂OMe | H | OCH₂CH₂OMe | NH |
| Me | Me | NHCO(CH₂)₂OMe | OH | H | NH |
| Me | Me | NHCO(CH₂)₂OMe | H | OH | NH |
| Me | Me | NHCO(CH₂)₂OMe | OMe | H | NH |
| Me | Me | NHCO(CH₂)₂OMe | H | OMe | NH |
| Me | Me | NHCO(CH₂)₂OMe | OEt | H | NH |
| Me | Me | NHCO(CH₂)₂OMe | H | OEt | NH |
| Me | Me | NHCO(CH₂)₂OMe | OCH₂CH₂OMe | H | NH |
| Me | Me | NHCO(CH₂)₂OMe | H | OCH₂CH₂OMe | NH |
| Me | Me | OCH₂OMe | OH | H | NH |
| Me | Me | OCH₂OMe | H | OH | NH |
| Me | Me | OCH₂OMe | OMe | H | NH |
| Me | Me | OCH₂OMe | H | OMe | NH |
| Me | Me | OCH₂OMe | OEt | H | NH |
| Me | Me | OCH₂OMe | H | OEt | NH |
| Me | Me | OCH₂OMe | OCH₂CH₂OMe | H | NH |
| Me | Me | OCH₂OMe | H | OCH₂CH₂OMe | NH |
| Me | Me | O(CH₂)₂OMe | OH | H | NH |
| Me | Me | O(CH₂)₂OMe | H | OH | NH |
| Me | Me | O(CH₂)₂OMe | OMe | H | NH |
| Me | Me | O(CH₂)₂OMe | H | OMe | NH |
| Me | Me | O(CH₂)₂OMe | OEt | H | NH |
| Me | Me | O(CH₂)₂OMe | H | OEt | NH |
| Me | Me | O(CH₂)₂OMe | OCH₂CH₂OMe | H | NH |
| Me | Me | O(CH₂)₂OMe | H | OCH₂CH₂OMe | NH |
| Me | Me | CONH-cyclopropyl | OH | H | NH |
| Me | Me | CONH-cyclopropyl | H | OH | NH |
| Me | Me | CONH-cyclopropyl | OMe | H | NH |
| Me | Me | CONH-cyclopropyl | H | OMe | NH |
| Me | Me | CONH-cyclopropyl | OEt | H | NH |
| Me | Me | CONH-cyclopropyl | H | OEt | NH |
| Me | Me | CONH-cyclopropyl | OCH₂CH₂OMe | H | NH |
| Me | Me | CONH-cyclopropyl | H | OCH₂CH₂OMe | NH |
| Me | Me | H | OH | H | NH |
| Me | Me | H | H | OH | NH |
| Me | Me | H | OMe | H | NH |
| Me | Me | H | H | OMe | NH |
| Me | Me | H | OEt | H | NH |
| Me | Me | H | H | OEt | NH |
| Me | Me | H | OCH₂CH₂OMe | H | NH |
| Me | Me | H | H | OCH₂CH₂OMe | NH |
| Me | H | CH₂OH | OH | H | NH |
| Me | H | CH₂OH | H | OH | NH |
| Me | H | CH₂OH | OMe | H | NH |
| Me | H | CH₂OH | H | OMe | NH |
| Me | H | CH₂OH | OEt | H | NH |
| Me | H | CH₂OH | H | OEt | NH |
| Me | H | CH₂OH | OCH₂CH₂OMe | H | NH |
| Me | H | CH₂OH | H | OCH₂CH₂OMe | NH |
| Me | H | CH₂OCH₃ | OH | H | NH |
| Me | H | CH₂OCH₃ | H | OH | NH |
| Me | H | CH₂OCH₃ | OMe | H | NH |
| Me | H | CH₂OCH₃ | H | OMe | NH |
| Me | H | CH₂OCH₃ | OEt | H | NH |
| Me | H | CH₂OCH₃ | H | OEt | NH |
| Me | H | CH₂OCH₃ | OCH₂CH₂OMe | H | NH |
| Me | H | CH₂OCH₃ | H | OCH₂CH₂OMe | NH |
| Me | H | CONHMe | OH | H | NH |
| Me | H | CONHMe | H | OH | NH |
| Me | H | CONHMe | OMe | H | NH |
| Me | H | CONHMe | H | OMe | NH |
| Me | H | CONHMe | OEt | H | NH |
| Me | H | CONHMe | H | OEt | NH |
| Me | H | CONHMe | OCH₂CH₂OMe | H | NH |
| Me | H | CONHMe | H | OCH₂CH₂OMe | NH |
| Me | H | CON-pyrrolidine | OH | H | NH |
| Me | H | CON-pyrrolidine | H | OH | NH |
| Me | H | CON-pyrrolidine | OMe | H | NH |
| Me | H | CON-pyrrolidine | H | OMe | NH |
| Me | H | CON-pyrrolidine | OEt | H | NH |
| Me | H | CON-pyrrolidine | H | OEt | NH |
| Me | H | CON-pyrrolidine | OCH₂CH₂OMe | H | NH |
| Me | H | CON-pyrrolidine | H | OCH₂CH₂OMe | NH |
| Me | H | CONH(CH₂)₂OH | OH | H | NH |
| Me | H | CONH(CH₂)₂OH | H | OH | NH |
| Me | H | CONH(CH₂)₂OH | OMe | H | NH |
| Me | H | CONH(CH₂)₂OH | H | OMe | NH |
| Me | H | CONH(CH₂)₂OH | OEt | H | NH |
| Me | H | CONH(CH₂)₂OH | H | OEt | NH |
| Me | H | CONH(CH₂)₂OH | OCH₂CH₂OMe | H | NH |
| Me | H | CONH(CH₂)₂OH | H | OCH₂CH₂OMe | NH |
| Me | H | CONH(CH₂)₂OMe | OH | H | NH |
| Me | H | CONH(CH₂)₂OMe | H | OH | NH |
| Me | H | CONH(CH₂)₂OMe | OMe | H | NH |
| Me | H | CONH(CH₂)₂OMe | H | OMe | NH |
| Me | H | CONH(CH₂)₂OMe | OEt | H | NH |
| Me | H | CONH(CH₂)₂OMe | H | OEt | NH |
| Me | H | CONH(CH₂)₂OMe | OCH₂CH₂OMe | H | NH |
| Me | H | CONH(CH₂)₂OMe | H | OCH₂CH₂OMe | NH |
| Me | H | CONH₂ | OH | H | NH |
| Me | H | CONH₂ | H | OH | NH |
| Me | H | CONH₂ | OMe | H | NH |
| Me | H | CONH₂ | H | OMe | NH |
| Me | H | CONH₂ | OEt | H | NH |
| Me | H | CONH₂ | H | OEt | NH |
| Me | H | CONH₂ | OCH₂CH₂OMe | H | NH |
| Me | H | CONH₂ | H | OCH₂CH₂OMe | NH |
| Me | H | CON-morpholine | OH | H | NH |
| Me | H | CON-morpholine | H | OH | NH |
| Me | H | CON-morpholine | OMe | H | NH |
| Me | H | CON-morpholine | H | OMe | NH |
| Me | H | CON-morpholine | OEt | H | NH |
| Me | H | CON-morpholine | H | OEt | NH |
| Me | H | CON-morpholine | OCH₂CH₂OMe | H | NH |
| Me | H | CON-morpholine | H | OCH₂CH₂OMe | NH |
| Me | H | CONMe₂ | OH | H | NH |
| Me | H | CONMe₂ | H | OH | NH |
| Me | H | CONMe₂ | OMe | H | NH |
| Me | H | CONMe₂ | H | OMe | NH |
| Me | H | CONMe₂ | OEt | H | NH |
| Me | H | CONMe₂ | H | OEt | NH |
| Me | H | CONMe₂ | OCH₂CH₂OMe | H | NH |
| Me | H | CONMe₂ | H | OCH₂CH₂OMe | NH |
| Me | H | CH₂O(CH₂)₂OMe | OH | H | NH |
| Me | H | CH₂O(CH₂)₂OMe | H | OH | NH |
| Me | H | CH₂O(CH₂)₂OMe | OMe | H | NH |
| Me | H | CH₂O(CH₂)₂OMe | H | OMe | NH |
| Me | H | CH₂O(CH₂)₂OMe | OEt | H | NH |
| Me | H | CH₂O(CH₂)₂OMe | H | OEt | NH |
| Me | H | CH₂O(CH₂)₂OMe | OCH₂CH₂OMe | H | NH |
| Me | H | CH₂O(CH₂)₂OMe | H | OCH₂CH₂OMe | NH |
| Me | H | CON-aziridine | OH | H | NH |
| Me | H | CON-aziridine | H | OH | NH |
| Me | H | CON-aziridine | OMe | H | NH |
| Me | H | CON-aziridine | H | OMe | NH |
| Me | H | CON-aziridine | OEt | H | NH |
| Me | H | CON-aziridine | H | OEt | NH |
| Me | H | CON-aziridine | OCH₂CH₂OMe | H | NH |
| Me | H | CON-aziridine | H | OCH₂CH₂OMe | NH |
| Me | H | COOEt | OH | H | NH |
| Me | H | COOEt | H | OH | NH |
| Me | H | COOEt | OMe | H | NH |
| Me | H | COOEt | H | OMe | NH |
| Me | H | COOEt | OEt | H | NH |
| Me | H | COOEt | H | OEt | NH |
| Me | H | COOEt | OCH₂CH₂OMe | H | NH |
| Me | H | COOEt | H | OCH₂CH₂OMe | NH |
| Me | H | COOH | OH | H | NH |
| Me | H | COOH | H | OH | NH |
| Me | H | COOH | OMe | H | NH |
| Me | H | COOH | H | OMe | NH |
| Me | H | COOH | OEt | H | NH |
| Me | H | COOH | H | OEt | NH |
| Me | H | COOH | OCH₂CH₂OMe | H | NH |
| Me | H | COOH | H | OCH₂CH₂OMe | NH |
| Me | H | CON-azetidine | OH | H | NH |
| Me | H | CON-azetidine | H | OH | NH |
| Me | H | CON-azetidine | OMe | H | NH |
| Me | H | CON-azetidine | H | OMe | NH |
| Me | H | CON-azetidine | OEt | H | NH |
| Me | H | CON-azetidine | H | OEt | NH |
| Me | H | CON-azetidine | OCH₂CH₂OMe | H | NH |
| Me | H | CON-azetidine | H | OCH₂CH₂OMe | NH |
| Me | H | CONH(CH₂)₂Me | OH | H | NH |
| Me | H | CONH(CH₂)₂Me | H | OH | NH |
| Me | H | CONH(CH₂)₂Me | OMe | H | NH |
| Me | H | CONH(CH₂)₂Me | H | OMe | NH |
| Me | H | CONH(CH₂)₂Me | OEt | H | NH |
| Me | H | CONH(CH₂)₂Me | H | OEt | NH |
| Me | H | CONH(CH₂)₂Me | OCH₂CH₂OMe | H | NH |
| Me | H | CONH(CH₂)₂Me | H | OCH₂CH₂OMe | NH |
| Me | H | CONHCH₂CHOHCH₂OH | OH | H | NH |
| Me | H | CONHCH₂CHOHCH₂OH | H | OH | NH |
| Me | H | CONHCH₂CHOHCH₂OH | OMe | H | NH |
| Me | H | CONHCH₂CHOHCH₂OH | H | OMe | NH |
| Me | H | CONHCH₂CHOHCH₂OH | OEt | H | NH |
| Me | H | CONHCH₂CHOHCH₂OH | H | OEt | NH |
| Me | H | CONHCH₂CHOHCH₂OH | OCH₂CH₂OMe | H | NH |
| Me | H | CONHCH₂CHOHCH₂OH | H | OCH₂CH₂OMe | NH |
| Me | H | NCH₃COCH₃ | OH | H | NH |
| Me | H | NCH₃COCH₃ | H | OH | NH |
| Me | H | NCH₃COCH₃ | OMe | H | NH |
| Me | H | NCH₃COCH₃ | H | OMe | NH |
| Me | H | NCH₃COCH₃ | OEt | H | NH |
| Me | H | NCH₃COCH₃ | H | OEt | NH |
| Me | H | NCH₃COCH₃ | OCH₂CH₂OMe | H | NH |
| Me | H | NCH₃COCH₃ | H | OCH₂CH₂OMe | NH |
| Me | H | NHCOCH₃ | OH | H | NH |
| Me | H | NHCOCH₃ | H | OH | NH |
| Me | H | NHCOCH₃ | OMe | H | NH |
| Me | H | NHCOCH₃ | H | OMe | NH |
| Me | H | NHCOCH₃ | OEt | H | NH |
| Me | H | NHCOCH₃ | H | OEt | NH |
| Me | H | NHCOCH₃ | OCH₂CH₂OMe | H | NH |
| Me | H | NHCOCH₃ | H | OCH₂CH₂OMe | NH |
| Me | H | NHCOCH₂OMe | OH | H | NH |
| Me | H | NHCOCH₂OMe | H | OH | NH |
| Me | H | NHCOCH₂OMe | OMe | H | NH |
| Me | H | NHCOCH₂OMe | H | OMe | NH |
| Me | H | NHCOCH₂OMe | OEt | H | NH |
| Me | H | NHCOCH₂OMe | H | OEt | NH |
| Me | H | NHCOCH₂OMe | OCH₂CH₂OMe | H | NH |
| Me | H | NHCOCH₂OMe | H | OCH₂CH₂OMe | NH |
| Me | H | NHCO(CH₂)₂OMe | OH | H | NH |
| Me | H | NHCO(CH₂)₂OMe | H | OH | NH |
| Me | H | NHCO(CH₂)₂OMe | OMe | H | NH |
| Me | H | NHCO(CH₂)₂OMe | H | OMe | NH |
| Me | H | NHCO(CH₂)₂OMe | OEt | H | NH |
| Me | H | NHCO(CH₂)₂OMe | H | OEt | NH |
| Me | H | NHCO(CH₂)₂OMe | OCH₂CH₂OMe | H | NH |
| Me | H | NHCO(CH₂)₂OMe | H | OCH₂CH₂OMe | NH |
| Me | H | OCH₂OMe | OH | H | NH |
| Me | H | OCH₂OMe | H | OH | NH |
| Me | H | OCH₂OMe | OMe | H | NH |
| Me | H | OCH₂OMe | H | OMe | NH |
| Me | H | OCH₂OMe | OEt | H | NH |
| Me | H | OCH₂OMe | H | OEt | NH |
| Me | H | OCH₂OMe | OCH₂CH₂OMe | H | NH |
| Me | H | OCH₂OMe | H | OCH₂CH₂OMe | NH |
| Me | H | O(CH₂)₂OMe | OH | H | NH |
| Me | H | O(CH₂)₂OMe | H | OH | NH |
| Me | H | O(CH₂)₂OMe | OMe | H | NH |
| Me | H | O(CH₂)₂OMe | H | OMe | NH |
| Me | H | O(CH₂)₂OMe | OEt | H | NH |
| Me | H | O(CH₂)₂OMe | H | OEt | NH |
| Me | H | O(CH₂)₂OMe | OCH₂CH₂OMe | H | NH |
| Me | H | O(CH₂)₂OMe | H | OCH₂CH₂OMe | NH |
| Me | H | CONH-cyclopropyl | OH | H | NH |
| Me | H | CONH-cyclopropyl | H | OH | NH |
| Me | H | CONH-cyclopropyl | OMe | H | NH |
| Me | H | CONH-cyclopropyl | H | OMe | NH |
| Me | H | CONH-cyclopropyl | OEt | H | NH |
| Me | H | CONH-cyclopropyl | H | OEt | NH |
| Me | H | CONH-cyclopropyl | OCH₂CH₂OMe | H | NH |
| Me | H | CONH-cyclopropyl | H | OCH₂CH₂OMe | NH |
| Me | H | H | OH | H | NH |
| Me | H | H | H | OH | NH |
| Me | H | H | OMe | H | NH |
| Me | H | H | H | OMe | NH |
| Me | H | H | OEt | H | NH |
| Me | H | H | H | OEt | NH |
| Me | H | H | OCH₂CH₂OMe | H | NH |
| Me | H | H | H | OCH₂CH₂OMe | NH |

Exemplary particularly preferred compounds are those of the formula 2, in which R1, R2, R3, R4a, R4b and X have the meanings given in the following table 2 (Me=CH₃, Et=C₂H₅) and R5 and R6 are hydrogen, and the salts of these compounds.

TABLE 2

| R1 | R2 | R3 | R4a | R4b | X |
|---|---|---|---|---|---|
| Me | Me | H | OH | H | NH |
| Me | Me | H | H | OH | NH |
| Me | Me | H | OMe | H | NH |
| Me | Me | H | H | OMe | NH |
| Me | Me | H | OEt | H | NH |
| Me | Me | H | H | OEt | NH |
| Me | Me | H | OCH₂CH₂OMe | H | NH |
| Me | Me | H | H | OCH₂CH₂OMe | NH |
| Me | H | H | OH | H | NH |
| Me | H | H | H | OH | NH |
| Me | H | H | OMe | H | NH |
| Me | H | H | H | OMe | NH |
| Me | H | H | OEt | H | NH |
| Me | H | H | H | OEt | NH |
| Me | H | H | OCH₂CH₂OMe | H | NH |
| Me | H | H | H | OCH₂CH₂OMe | NH |
| Me | H | H | OH | H | O |
| Me | H | H | H | OH | O |
| Me | H | H | OMe | H | O |
| Me | H | H | H | OMe | O |
| Me | H | H | OEt | H | O |
| Me | H | H | H | OEt | O |
| Me | H | H | OCH₂CH₂OMe | H | O |
| Me | H | H | H | OCH₂CH₂OMe | O |
| Me | Me | H | OH | H | O |
| Me | Me | H | H | OH | O |
| Me | Me | H | OMe | H | O |
| Me | Me | H | H | OMe | O |
| Me | Me | H | OEt | H | O |
| Me | Me | H | H | OEt | O |
| Me | Me | H | OCH₂CH₂OMe | H | O |
| Me | Me | H | H | OCH₂CH₂OMe | O |
| Me | Me | CON(Me)₂ | OH | H | NH |
| Me | Me | CON(Me)₂ | H | OH | NH |
| Me | Me | CON(Me)₂ | OMe | H | NH |
| Me | Me | CON(Me)₂ | H | OMe | NH |
| Me | Me | CON(Me)₂ | OEt | H | NH |
| Me | Me | CON(Me)₂ | H | OEt | NH |
| Me | Me | CON(Me)₂ | OCH₂CH₂OMe | H | NH |
| Me | Me | CON(Me)₂ | H | OCH₂CH₂OMe | NH |
| Me | Me | CON(Me)₂ | O-n-butyl | H | NH |
| Me | Me | CON(Me)₂ | H | O-n-butyl | NH |
| Me | H | CON(Me)₂ | OH | H | NH |
| Me | H | CON(Me)₂ | H | OH | NH |
| Me | H | CON(Me)₂ | OMe | H | NH |
| Me | H | CON(Me)₂ | H | OMe | NH |
| Me | H | CON(Me)₂ | OEt | H | NH |
| Me | H | CON(Me)₂ | H | OEt | NH |
| Me | H | CON(Me)₂ | OCH₂CH₂OMe | H | NH |
| Me | H | CON(Me)₂ | H | OCH₂CH₂OMe | NH |
| Me | Me | CONHMe | OH | H | NH |
| Me | Me | CONHMe | H | OH | NH |
| Me | Me | CONHMe | OMe | H | NH |
| Me | Me | CONHMe | H | OMe | NH |
| Me | Me | CONHMe | OEt | H | NH |
| Me | Me | CONHMe | H | OEt | NH |
| Me | Me | CONHMe | OCH₂CH₂OMe | H | NH |
| Me | Me | CONHMe | H | OCH₂CH₂OMe | NH |
| Me | Me | CONHMe | O-n-butyl | H | NH |
| Me | Me | CONHMe | H | O-n-butyl | NH |
| Me | H | CONHMe | OH | H | NH |
| Me | H | CONHMe | H | OH | NH |
| Me | H | CONHMe | OMe | H | NH |
| Me | H | CONHMe | H | OMe | NH |
| Me | H | CONHMe | OEt | H | NH |
| Me | H | CONHMe | H | OEt | NH |
| Me | H | CONHMe | OCH₂CH₂OMe | H | NH |
| Me | H | CONHMe | H | OCH₂CH₂OMe | NH |
| Me | Me | CON(Me)₂ | OH | H | O |
| Me | Me | CON(Me)₂ | H | OH | O |
| Me | Me | CON(Me)₂ | OMe | H | O |
| Me | Me | CON(Me)₂ | H | OMe | O |
| Me | Me | CON(Me)₂ | OEt | H | O |
| Me | Me | CON(Me)₂ | H | OEt | O |
| Me | Me | CON(Me)₂ | OCH₂CH₂OMe | H | O |
| Me | Me | CON(Me)₂ | H | OCH₂CH₂OMe | O |
| Me | Me | CON(Me)₂ | O-n-butyl | H | O |
| Me | Me | CON(Me)₂ | H | O-n-butyl | O |
| Me | H | CON(Me)₂ | OH | H | O |
| Me | H | CON(Me)₂ | H | OH | O |
| Me | H | CON(Me)₂ | OMe | H | O |
| Me | H | CON(Me)₂ | H | OMe | O |
| Me | H | CON(Me)₂ | OEt | H | O |
| Me | H | CON(Me)₂ | H | OEt | O |
| Me | H | CON(Me)₂ | OCH₂CH₂OMe | H | O |
| Me | H | CON(Me)₂ | H | OCH₂CH₂OMe | O |
| Me | Me | H | OH | CH₃ | NH |
| Me | Me | H | CH₃ | OH | NH |
| Me | Me | H | OH | CH₂CH₃ | NH |
| Me | Me | H | CH₂CH₃ | OH | NH |
| Me | Me | H | OH | CH₃ | O |
| Me | Me | H | CH₃ | OH | O |
| Me | Me | H | OH | CH₂CH₃ | O |
| Me | Me | H | CH₂CH₃ | OH | O |

Particularly preferred are the compounds given as final products of formula 1 in the examples, and the salts of these compounds.

The compounds according to the invention can be synthesized from corresponding starting compounds, like the cyclic ketones (4) and (5), which can be prepared according to the reaction schemes given below (scheme 1 and scheme 2a and scheme 2b). The compounds of formula (1) where R4a or R4b is hydroxy are obtained by reduction of the compounds of formula (4) or (5) (X=NH or O) with for example sodium borohydride or any other reducing agent. The further synthesis of the compounds of formula (1), where R4a or R4b denotes alkoxy or alkoxyalkoxy, is then achieved by etherification of the compounds of formula (1), where R4a or R4b is hydroxy, under acidic conditions as shown in scheme 3. Alternatively, compounds of the formula (1) where one of the substituents R4a or R4b denotes alkyl and the other hydroxy are obtained by reacting compounds of the formula (4) or (5) (X=NH or O) with Grignard reagents as depicted in scheme 4.

### Scheme 1:

Preparation of compounds of the formula (4) where X=NH, with any desired substituent R1, R2, R3 and Ar:

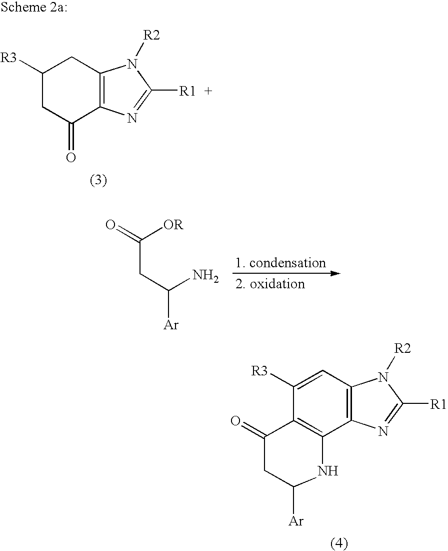

Preparation of compounds of the formula (5) where X=O, with any desired substituent R1, R2, R3 and Ar:

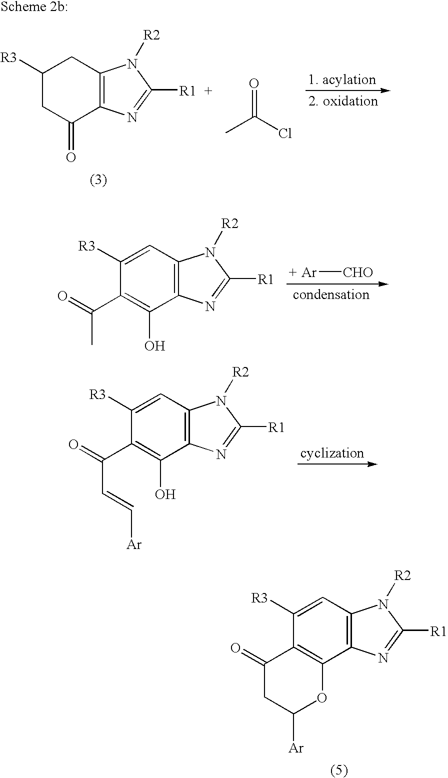

Alternative preparation of compounds of the formula (5) where X=O, with any desired substituent R1, R2, R3 and Ar:

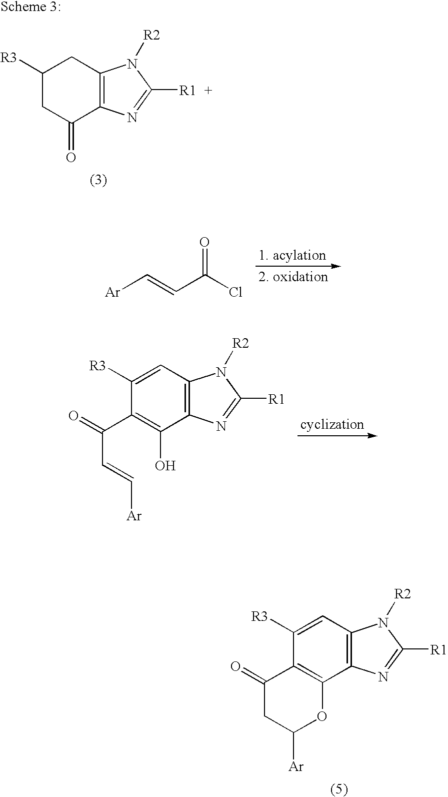

Preparation of compounds of the formula (1) where X=NH or O and R4a or R4b are hydroxy, 1-4C-alkoxy or 1-4C-alkoxy-1-4C-alkoxy with any desired substituent R1, R2, R3 and Ar:

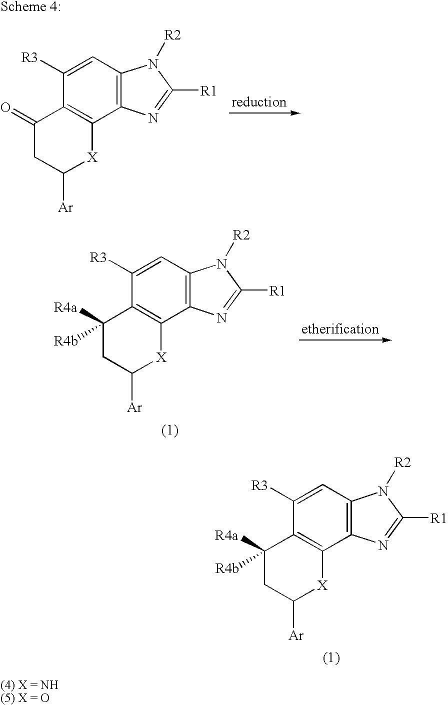

Preparation of compounds of the formula (1) where X=NH or O and one of the substituents R4a or R4b denotes 1-4C-alkyl and the other hydroxy with any desired substituent R1, R2, R3 and Ar:

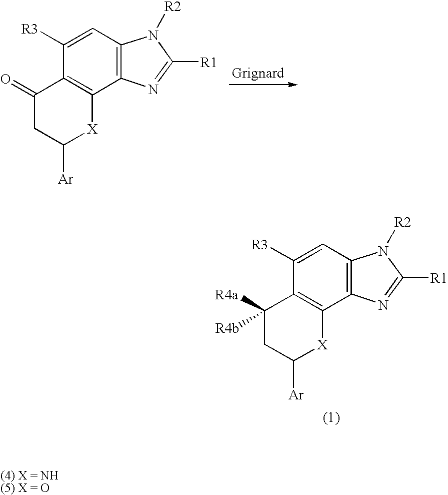

Alternatively, the compounds of the formula 1 according to the invention where X=NH can be synthesized from corresponding starting compounds, like the cyclic ketones (7), which can be prepared according to the reaction scheme given below (scheme 5).

In a first step ketones of the formula (3) are reacted with protected phenylisoserine derivatives (wherein Y is a suitable leaving group, for example an ethoxy group and Prot is a suitable protecting group like a suitable silyl radical, for example a ^{t}BuMe₂Si-radical) to give compounds of the formula (6) and/or compounds of the formula (6a). Compounds of the formula 6a, if obtained, can be re-protected by standard procedures to the desired compounds of the formula (6). The subsequent oxidation delivers cyclic ketones of formula (7). The synthesis of protected phenylisoserine derivatives used in this reaction sequence can be performed for example as described in the International Patent Application WO 05/058893.

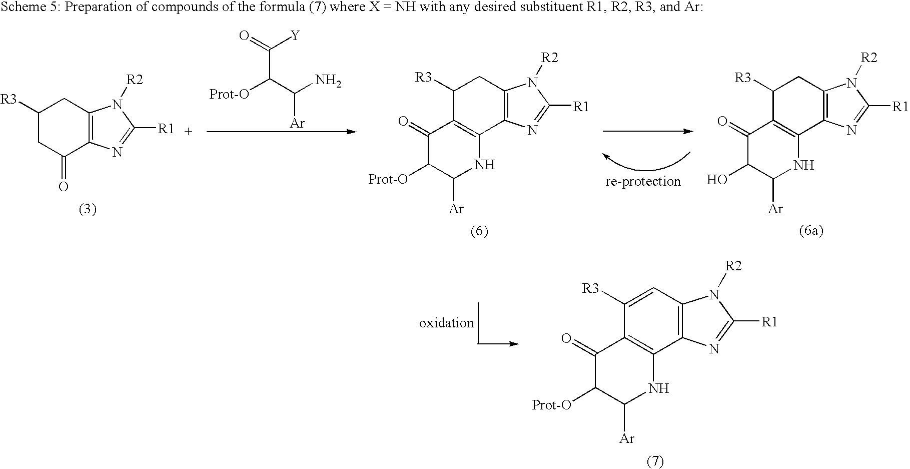

The compounds of formula (1) where X=NH and R4a or R4b is hydroxy are obtained as outlined in route A of reaction scheme 6:

Reduction of the compounds of formula (7) with for example sodium borohydride or any other reducing agent forms alcohol (8), in which the hydroxyl group can be protected by a suitable protecting group pg (e.g. tert.-butyldimethylsilyl group), removal of the protecting group Prot (e.g. the acetyl group), deoxygenation of the resulting alcohol [by methodologies known to the expert, for example the Barton-McCombie deoxygenation methodology using a base like diisopropylethylamine, methyl iodide, and carbon disulfide followed by tributyltin hydride and 2,2′-azobis(isobutyronitrile) as described by D. H. R. Barton, S. W. McCombie, J. Chem. Soc., Perkin Trans. 1, (1975), 1574], and finally removal of the protecting group pg delivers compounds of formula (1) where X=NH and R4a or R4b is hydroxyl. The compounds of formula (1), where X=NH and R4a or R4b denotes alkoxy or alkoxyalkoxy, can be synthesized for example as outlined in route B of reaction scheme 6. Alcohols of formula (8) can be etherified under acidic conditions. The protecting group Prot can be removed before or after the etherification step (in scheme 6 the deprotection after the etherification is shown). Deoxygenation of the resulting alcohol [by methodologies as described above or by a modified methodology using hypophosphorous acid as reducing agent as described by E. Lee, H. O. Han, Tetrah. Lett., (2002), 43, 7295-7296] formes the compounds of formula (1), where X=NH and R4a or R4b denotes alkoxy or alkoxyalkoxy.

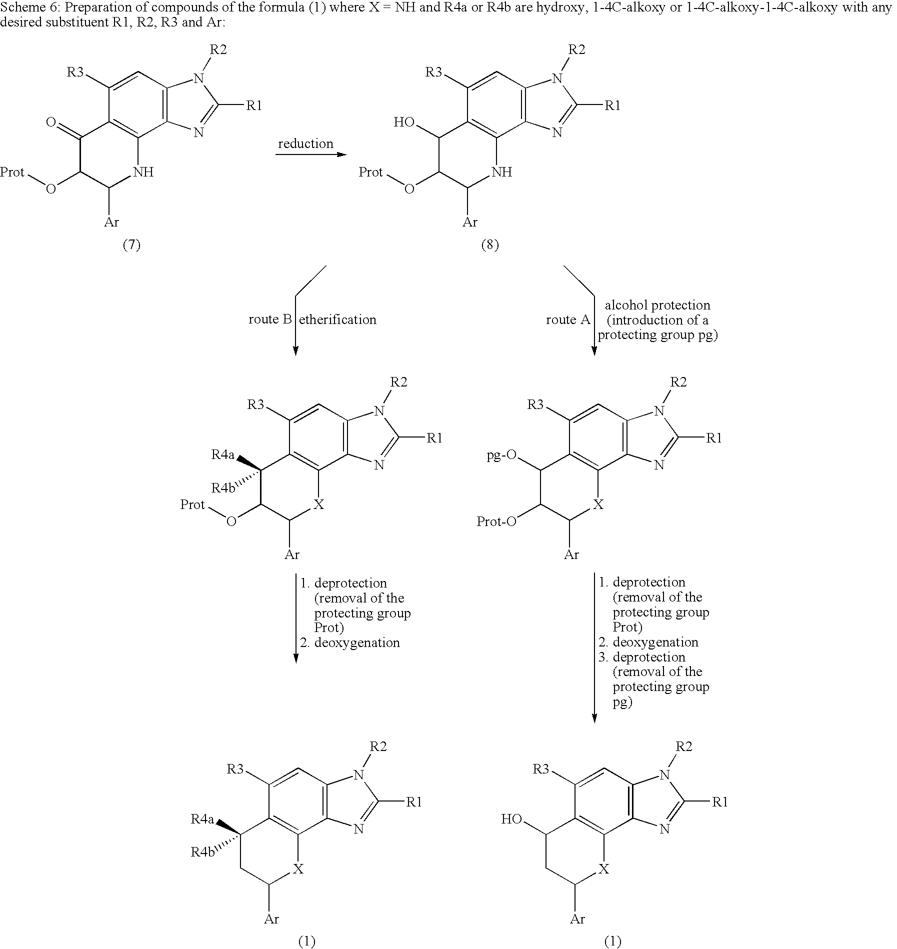

The starting materials from schemes 1, 2a, 2b, and 5—compounds of the formula (3)—are known for example from Helvetica Chimica Acta, 1979, 62, 507 or from WO 05/058893 or can be prepared in an analogous manner as described therein.

The starting materials from schemes 1, 2a, 2b, and 5—compounds of the formula (3)—can alternatively be prepared as shown, for example, in scheme 7 (route A) performing the cyclization reaction of compounds of the formula (11) in the presence of a primary amine (R2≠H) or ammonia (R2=H). The preparation of compounds of the formula (11) can be achieved by several methodologies known to the expert; two examples are illustrated in scheme 7 (route A):

The reduction of azo-compounds of the formula (9) by methodologies which are performed in a manner known to the expert, for example as described by A. Treibs, R. Zinsmeister in Chem. Ber. (1957), 90, 85-87, followed by an acylation reaction delivers compounds of formula (11). Alternatively, aromatic compounds of the formula (10) can be reduced by strong reducing agents followed by an acidic workup, for example as described by Kuehne, Lambert in Org. Synth.; Coll. Vol. V, (1973), 400 or by A. Mann, C. Humblet in J. Med. Chem., (1985), 28, 1440-1446. Compounds of the formula (9) and (11) are known, for example from the French Patent FR2242984, or from Allan, Collect. Czech. Chem. Commun. (1966), 31, 4129, or they can be prepared using analogues process steps.

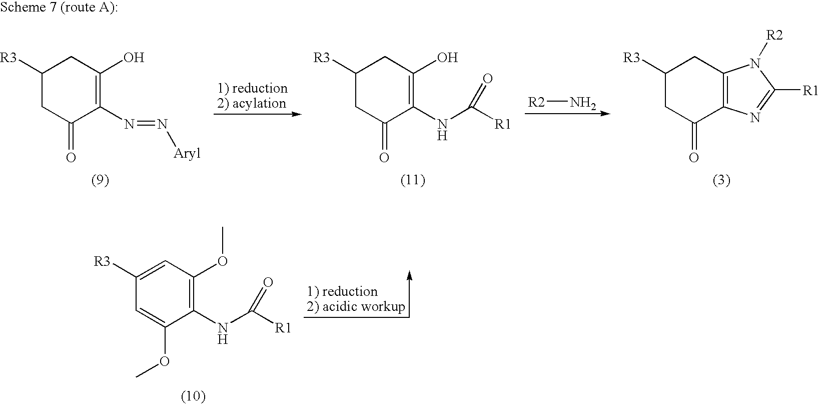

Alternatively, the starting materials from schemes 1, 2a, 2b, and 5—compounds of the formula (3)—can be prepared in a manner as shown for example in scheme 7 (route B). Compounds of the formula (12) can be hydrogenated to the desired compounds of the formula (3) in manner known to the expert, for example as described by H. Oelschlaeger and H. Giebenhain in Archiv der Pharmazie, 1973, 306, 485-489. The starting compounds of the formula 8 are known, for example, from A. R. Katritzky et al., Heterocycles (1995), 41, 345-352 or from WO 04/054984 or they can be prepared using analogous process steps.

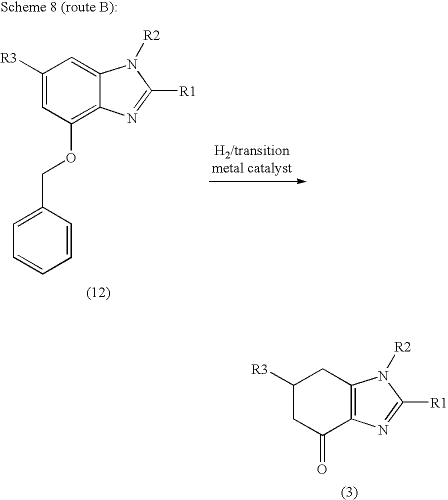

The derivatization, if any, of the compounds obtained according to the above Schemes 1 to 6 (e.g. conversion of a group R3 into another group R3, or of R2=H into another group e.g. R2=1-4C-alkyl) is likewise carried out in a manner known to the expert. If, for example, compounds where R3=—CO-1-4C-alkoxy or R3=—CO—NR31R32 are desired, an appropriate derivatization can be performed in a manner known to the expert (e.g. metal catalysed carbonylation of the corresponding halo compound or conversion of an ester into an amide), e.g. at the stage of the benzimidazoles given in schemes 1, 2, or 5 or more conveniently at a later point in time.

The compounds of the formula 2 can be isolated from the corresponding racemic mixtures of the formula 1 by techniques known to the expert. The separation can be achieved by methods known to the expert, for example by preparative chromatography using a chiral column.

The following examples serve to illustrate the invention in greater detail without restricting it. Likewise, further compounds of the formula 1, 1a or 2, for example those from the tables 1 and 2, whose preparation is not described explicitly can be prepared in an analogous manner or in a manner familiar per se to the person skilled in the art using customary process techniques. The abbreviation min stands for minute(s), h for hour(s).

### EXAMPLES

### I. Final Products of Formula 1

### 1. cis-2,3-Dimethyl-8-phenyl-3,6,7,8-tetrahydro-chromeno[7,8-d]imidazol-6-ol

To a solution of 0.2 g (0.7 mmol) 2,3-dimethyl-8-phenyl-7,8-dihydro-3H-chromeno[7,8-d]imidazol-6-one in 5 ml methanol were added 0.05 g (1.3 mmol) sodium borohydride in small portions. After 30 min, the reaction mixture was hydrolyzed with saturated aqueous ammonium chloride and extracted with dichloromethane. The combined organic phases were dried over anhydrous magnesium sulphate and evaporated. Purification of the residue by crystallization from diethyl ether yielded 0.19 g (95%) of the title compound as a solid (m.p. 228-229° C.).

### 2. 6-(2-Methoxy-ethoxy)-2,3-dimethyl-8-phenyl-3,6,7,8-tetrahydro-chromeno[7,8-d]imidazole (cis/trans 6:4)

To a solution of 0.2 g (0.7 mmol) cis-2,3-dimethyl-8-phenyl-3,6,7,8-tetrahydro-chromeno[7,8-d]imidazol-6-ol in 2 ml 2-methoxy-ethanol were added 0.12 ml (1.9 mmol) methanesulphonic acid and the mixture was stirred at 60° C. After 2 h, the reaction mixture was cooled down, neutralized with saturated aqueous sodium bicarbonate and extracted with dichloromethane. The combined organic phases were dried over anhydrous magnesium sulphate and evaporated. Purification of the residue by column chromatography (silica gel, ethyl acetate) and crystallization from diethyl ether yielded 0.04 g (17%) of the title compound as a mixture of diastereomers cis/trans 6:4 (m.p. 148-149° C.).

### 3. 6-(2-Ethoxy)-2,3-dimethyl-8-phenyl-3,6,7,8-tetrahydro-chromeno[7,8-d]imidazole

To a solution of 0.3 g (1 mmol) cis-2,3-dimethyl-8-phenyl-3,6,7,8-tetrahydro-chromeno[7,8-d]imidazol-6-ol in 3 ml ethanol were added 0.2 ml (2.5 mmol) methanesulphonic acid and the mixture was stirred at 60° C. After 4 h, the reaction mixture was cooled down, neutralized with saturated aqueous sodium bicarbonate and extracted with dichloromethane. The combined organic phases were dried over anhydrous magnesium sulphate and evaporated. Purification of the residue by crystallization from ethyl acetate yielded 0.2 g (61%) of the title compound (m.p. 177-178° C.).

### 4. (6R,8S)-6-n-Butoxy-2,3-dimethyl-8-phenyl-6,7,8,9-tetrahydro-3H-imidazo[4,5-h]quinoline-5-carboxylic acid dimethylamide

### Thiocarbonic acid O-((6S,7R,8R)-6-n-butoxy-5-dimethylcarbamoyl-2,3-dimethyl-8-phenyl-6,7,8,9-tetrahydro-3H-imidazo[4,5-h]quinolin-7-yl) ester O-phenyl ester

Pyridine (88 μl, 90 mg, 1.14 mmol) and N,N-dimethylaminopyridine (20 mg, 0.16 mmol) were added to a solution of (6S,7R,8R)-[6-n-butyloxy-7-hydroxy-2,3-dimethyl-8-phenyl-6,7,8,9-tetrahydro-3H-imidazo[4,5-h]quinoline-5-yl]-carboxylic acid dimethylamide (0.25 g, 0.57 mmol) in dichloromethane (4 ml), and the reaction mixture was cooled to 0° C. The solution of phenyl chlorothionoformate (0.132 ml, 0.168 g, 0.973 mmol) in dichloromethane (1 ml) was added dropwise. After stirring for 30 min at 0° C. and 2 h at room temperature, the reaction mixture was poured onto an aqueous saturated solution of sodium bicarbonate, the phases were separated, and the aqueous phase was extracted with dichloromethane. The combined organic phases were washed with water, dried (MgSO₄), and the solvent was removed to obtain 0.23 g of a yellow oil as crude product which was used in the next step without purification. MS (ESI): 573.2 (MH⁺).

### (6R,8S)-6-n-Butoxy-2,3-dimethyl-8-phenyl-6,7,8,9-tetrahydro-3H-imidazo[4,5-h]quinoline-5-carboxylic acid dimethylamide

Thiocarbonic acid O-((6S,7R,8R)-6-n-butoxy-5-dimethylcarbamoyl-2,3-dimethyl-8-phenyl-6,7,8,9-tetrahydro-3H-imidazo[4,5-h]quinolin-7-yl) ester O-phenyl ester (crude product, 0.23 g, 0.40 mmol) was dissolved in dioxane (2.5 ml). Hypophosphorous acid (50 wt % in water) (0.22 ml, 2.12 mmol) and triethylamine (0.28 ml, 0.22 g, 2.2 mmol) were added and the solution was heated to 100° C. Over a period of 20 min 2,2′-azobis(isobutyronitrile) (66 mg, 0.40 mmol) was added. The reaction mixture was stirred for 60 min at 100° C., further 2,2′-azobis(isobutyronitrile) (33 mg, 0.20 mmol) was added, and the heating was continued for further 2 h. The reaction mixture was cooled to room temperature and poured onto an aqueous saturated solution of sodium bicarbonate. The phases were separated and the aqueous phase was extracted with dichloromethane. The combined organic phases were washed with water, dried (MgSO₄), and the solvent was removed. Purification of the crude product was carried out by flash column chromatography (silica gel, dichloromethane/methanol 100/1, then 100/3) delivering after crystallization from diisopropyl ether/ethyl acetate 16 mg (1%) of the title compound as yellow crystals.

¹H-NMR (200 MHz, CD₂Cl₂): δ [ppm]=0.97 (t, 3H), 1.29-1.68 (m, 4H), 1.83-1.96 (m, 1H), 2.25-2.40 (m, 1H), 2.53 (s, 3H), 2.91 (s, 3H), 3.13 (s, 3H), 3.28-3.52 (m, 1H), 3.58-3.82 (m, 1H), 4.57 (d, 1H), 4.69 (s, 1H), 5.30 (s, 1H), 6.52 (s, 1H), 7.25-7.68 (m, 5H).

### II. Starting Compounds and Intermediates

### A. 1-(4-Hydroxy-1,2-dimethyl-6,7-dihydro-1H-benzolmidazol-5-yl)-3-phenyl-propenone

To a suspension of 5.0 g (30.4 mmol) 1,2-dimethyl-1,5,6,7-tetrahydro-benzoimidazol-4-one in 40 ml tetrahydrofuran were slowly added 61 ml (61 mmol) sodium bis(trimethylsilylamide) (1M in tetrahydrofuran) at −5° C. and the resulting red solution was stirred 1 h at ambient temperature. The mixture was cooled to −78° C. and a solution of 5.5 g (33 mmol) cinnamoyl chloride in 20 ml tetrahydrofuran was slowly added. After 1.5 h at −78° C., the reaction mixture was hydrolyzed with saturated aqueous ammonium chloride and partitioned between water and dichloromethane. The organic layer was separated, dried over anhydrous magnesium sulphate and evaporated. Purification of the residue by column chromatography (silica gel, dichloromethane/methanol 13/1) and crystallization from ethyl acetate/diethyl ether yielded 2.3 g (26%) of the title compound as a yellow solid (m.p. 216-217° C.).

### B. 1-(4-Hydroxy-1,2-dimethyl-1H-benzoimidazol-5-yl)-3-phenyl-propenone

To a stirred solution of 5.0 g (17 mmol) 1-(4-hydroxy-1,2-dimethyl-6,7-dihydro-1H-benzoimidazol-5-yl)-3-phenyl-propenone in 90 ml chloroform were added 15 g (173 mmol) manganese(IV) oxide. After 2 h at ambient temperature, a second amount of 15 g manganese(IV) oxide was added and stirring was continued for 2 h. The reaction mixture was filtered through celite and the solids were thoroughly washed with boiling dichloromethane/methanol. The filtrate was evaporated and the residue was purified by column chromatography (silica gel, ethyl acetate) to yield 2.35 g (47%) of the title compound as a yellow solid (m.p. 230-231° C.).

### C. 2,3-Dimethyl-8-phenyl-7,8-dihydro-3H-chromeno[7,8-d]imidazol-6-one

A mixture of 0.4 g (1.4 mmol) 1-(4-hydroxy-1,2-dimethyl-1H-benzoimidazol-5-yl)-3-phenyl-propenone in 10 ml acetic acid and 10 ml phosphoric acid was heated to 100° C. to give a yellow solution. After 1 h, the reaction mixture was poured onto crushed ice, neutralized with conc. ammonia and extracted with dichloromethane. The combined organic phases were dried over anhydrous magnesium sulphate and evaporated. Purification of the residue by column chromatography (silica gel, ethyl acetate) and crystallization from ethyl acetate yielded 0.3 g (75%) of the title compound as a yellow solid (m.p. 201-202° C.).

### D. (2R,3R)-3-Amino-2-(tert.-butyl-dimethyl-silanyloxy)-3-phenyl proplonic acid ethyl ester

(R,R)-Phenylisoserine ethyl ester (1323 g, 4.06 mole) was dissolved in dichloromethane (6.6 l). To this solution, imidazole (397.4 g) and t-butyldimethylsilyl chloride (724 g) were added. The mixture was stirred for 16 h at room temperature. The reaction mixture was washed subsequently with 6 l and 4 l of water. The resulting clear dichloromethane layer was dried over sodium sulphate, filtered and concentrated under reduced pressure. The obtained 1509 g of the title compound was used as such for the next reaction step without further purification.

### E. 3-Hydroxy-5-oxo-4-phenylazo-cyclohex-3-enecarboxylic acid dimethylamide

The suspension of 3-hydroxy-5-oxo-4-phenylazo-cyclohex-3-enecarboxylic acid (30.0 g, 115 mmol) and O-(1H-benzotriazol-1-yl)-N,N,N′,N′-tetramethyluronium tetrafluoroborate (TBTU) (38.9 g, 121 mmol) in dichloromethane (400 ml) was heated to reflux for 2 h. After cooling to room temperature, the solution of dimethylamine in tetrahydrofuran (2N) (575 ml, 1.15 mol) was added and the reaction mixture was stirred for 2 h at room temperature. Hydrochloric acid (2N) (500 ml) was added to acidify the reaction mixture (pH 1-2), and the aqueous phase was extracted with dichloromethane. The combined organic phases were washed with an aqueous solution of sodium bicarbonate, dried (MgSO₄), the solvent was removed, and the raw product was recrystallized from diisopropyl ether. 13.45 g (41%) of the title compound were isolated as pale yellow crystals (m.p. 179-181° C.).

### F. 4-Acetylamino-3-hydroxy-5-oxo-cyclohex-3-enecarboxylic acid dimethylamide

3-Hydroxy-5-oxo-4-phenylazo-cyclohex-3-en carboxylic acid dimethylamid (34.0 g, 118 mmol) was suspended in a mixture of acetic anhydride (68 ml, 710 mmol) and glacial acetic acid (340 ml). Zinc powder (46.4 g, 710 mmol) was added portionwise keeping the temperature under 60° C. The reaction mixture was stirred for 1 h, diluted with dioxan, filtered over a pad of silica gel, and concentrated. The residue was purified by flash column chromatography (silica gel, toluene/dioxan/methanol 6/3/1) to give, after crystallization from diisopropyl ether, 22.3 g (79%) of the title product as white crystals (m.p. 162-164° C.).

### G. 4,5,6,7-Tetrahydro-2,3-dimethyl-7-oxo-3H-benzimidazol-5-carboxylic acid dimethylamide

To a solution of 4-acetylamino-3-hydroxy-5-oxo-cyclohex-3-encarboxylic acid dimethylamide (3.5 g, 14.56 mmol) in toluene (25 ml) were added a solution of methylamine in tetrahydrofuran (2N) (15 ml, 30 mmol) and glacial acetic acid (3.5 ml). The reaction mixture was transferred into an autoclave and heated for 2 h at 180° C. After cooling down the solvent is removed, and the residue is purified by flash column chromatography (silica gel, dichloromethane/methanol 100/1, then 10/1) to give, after crystallization from diethyl ether, 2.5 g (73%) of the title product as white crystals (m.p. 190-194° C.).

### H. (7R,8R)-[7-Hydroxy-2,3-dimethyl-6-oxo-8-phenyl-4,5,6,7,8,9-hexahydro-3H-imidazo[4,5-h]quinoline-5-yl]-carboxylic acid dimethylamide

The suspension of 4,5,6,7-tetrahydro-2,3-dimethyl-7-oxo-3H-benzimidazol-5-carboxylic acid dimethylamide (8.5 g, 36.12 mmol) and (2R,3R)-3-amino-2-(tert.-butyl-dimethyl-silanyloxy)-3-phenyl propionic acid ethyl ester (12.3 g, 37.93 mmol) in 2-methoxyethanol (85 ml) was heated at 150° C. for 5 days, further (2R,3R)-3-amino-2-(tert.-butyl-dimethyl-silanyloxy)-3-phenyl propionic acid ethyl ester (3.00 g, 9.25 mmol) was added, and the reaction mixture was heated at 150° C. for further 2 days. After cooling down, the solvent was removed by distillation, the remaining oil was dissolved in dichloromethane and water, and the aqueous phase was extracted with dichloromethane. Washing of of the combined organic phases with water, drying (MgSO₄), removal of the solvent, and purification of the raw product by flash column chromatography (silica gel, dichloromethane/methanol 100/3, then 20/1) delivered after crystallization from diisopropyl ether 6.00 g (44%) of the title compound as white crystals (m.p. 224-226° C.).

### I. (7R,8R)-[7-Acetoxy-2,3-dimethyl-6-oxo-8-phenyl-4,5,6,7,8,9-hexahydro-3H-imidazo[4,5-h]quinoline-5-yl]-carboxylic acid dimethylamide

To the suspension of (7R,8R)-[7-hydroxy-2,3-dimethyl-6-oxo-8-phenyl-4,5,6,7,8,9-hexahydro-3H-imidazo[4,5-h]quinoline-5-yl]-carboxylic acid dimethylamide (9.00 g, 23.65 mmol) in acetic anhydride (90 ml) was added methane sulfonic acid (3.5 ml). A pale yellow solution was formed which was stirred for 2 h at room temperature. Acetic anhydride was removed by distillation, the remaining oil was dissolved in dichloromethane, and the solution was neutralized by addition of an aqueous solution of sodium bicarbonate (pH 8). Drying of the organic solution (MgSO₄), removal of the solvent, and purification by flash column chromatography (silica gel, dichloromethane/methanol 100/3) delivered a pale yellow oil which was crystallized from diisopropyl ether. 7.54 g (75%) of the title product were isolated as pale yellow crystals (m.p. 198-207° C.).

### J. (7R,8R)-[7-Acetoxy-2,3-dimethyl-6-oxo-8-phenyl-6,7,8,9-tetrahydro-3H-imidazo[4,5-h]quinoline-5-yl]-carboxylic acid dimethylamide

(7R,8R)-[7-Acetoxy-2,3-dimethyl-6-oxo-8-phenyl-4,5,6,7,8,9-hexahydro-3H-imidazo[4,5-h]quinoline-5-yl]-carboxylic acid dimethylamide (7.40 g, 17.51 mmol) were suspended in ethyl acetate (120 ml) and 2,3-dichloro-4,5-dicyano benzoquinone (7.95 g, 35.00 mmol) were added in portions at room temperature. A dark solution resulted which was stirred for 24 h at room temperature. The reaction mixture was poured onto an aqueous solution of sodium bicarbonate, the phases were separated, and the aqueous phase was extracted with dichloromethane. The combined organic phases were washed with water, dried (MgSO₄), the solvent was removed, and the raw product was purified by flash column chromatography (silica gel, toluene/dioxan/methanol 6/3.5/0.5). The title product was crystallized from diisopropyl ether yielding 5.03 g (68%) of yellow crystals (m.p. 219-222° C.).

### K. (7R,8R)-[7-Hydroxy-2,3-dimethyl-6-oxo-8-phenyl-6,7,8,9-tetrahydro-3H-imidazo[4,5-h]quinoline-5-yl]-carboxylic acid dimethylamide

Hydrazine hydrate (1.17 g, 23.3 mmol) were added at room temperature to the suspension of (7R,8R)-[7-acetoxy-2,3-dimethyl-6-oxo-8-phenyl-6,7,8,9-tetrahydro-3H-imidazo[4,5-h]quinoline-5-yl]-carboxylic acid dimethylamide (4.90 g, 11.65 mmol) in methanol (50 ml). The reaction mixture was stirred for 18 h at room temperature, poured onto water, and extracted with dichloromethane. The combined organic phases were washed with water, dried (MgSO₄), and the solvent was removed. A yellow oil resulted which was crystallized from diisopropyl ether yielding 4.29 g (97%) of yellow crystals.

¹H-NMR (200 MHz, d₆-DMSO): δ [ppm]=2.50 (s, 3H), 2.60 (d, 3H), 2.94 (d, 3H), 3.69 (s, 3H), 4.11-4.28 (m, 1H), 4.59 (d, 2H), 5.51 (dd, 1H), 6.65 (d, 1H), 6.96 (d, 1H), 7.23-7.47 (m, 5H).

### L. (7R,8R)-[6,7-Dihydroxy-2,3-dimethyl-8-phenyl-6,7,8,9-tetrahydro-3H-imidazo[4,5-h]quinoline-5-yl]-carboxylic acid dimethylamide

To a at 0° C. cooled stirred solution of (7R,8R)-[7-hydroxy-2,3-dimethyl-6-oxo-8-phenyl-6,7,8,9-tetrahydro-3H-imidazo[4,5-h]quinoline-5-yl]-carboxylic acid dimethylamide (3.50 g, 9.24 mmol) in methanol (50 ml) was added sodium borohydride (0.70 g, 18.49 mmol). The reaction mixture was stirred for 1 h at 0° C. and 5 h at room temperature, and further 18 h at 0° C. Water and a saturated ammonium chloride solution were added to neutralize (pH7-8), the aqueous phase was extracted with dichloromethane twice, the combined organic layers were washed with a small amount of water, dried (MgSO₄), and concentrated in vacuo. Purification by flash column chromatography (silica gel, dichloromethane/methanol 100/3) and crystallization from diisopropyl ether gave 1.97 g (56%) of the title product as diastereomeric mixture that was not separable. MS (ESI): 381.2 (MH⁺).

### M. (6S,7R,8R)-[6-n-Butyloxy-7-hydroxy-2,3-dimethyl-8-phenyl-6,7,8,9-tetrahydro-3H-imidazo[4,5-h]quinoline-5-yl]-carboxylic acid dimethylamide

To a at −30° C. cooled stirred solution of (7R,8R)-[6,7-dihydroxy-2,3-dimethyl-8-phenyl-6,7,8,9-tetrahydro-3H-imidazo[4,5-h]quinoline-5-yl]-carboxylic acid dimethylamide (0.55 g, 1.44 mmol) in a mixture of n-butanol (8 ml) and dichloromethane (4 ml) was added dropwise methansulfonic acid (0.18 ml, 1.87 mmol). The reaction mixture was stirred for 30 min at −30° C., warmed to room temperature, and stirred at room temperature for 3 h. The solution was poured onto water, neutralized with a saturated sodium hydrogen carbonate solution (pH 8), and extracted with dichloromethane. The combined organic layers were washed with water, dried (MgSO₄), concentrated in vacuo, and purified by flash column chromatography (silica gel, dichloromethane/methanol 100/3) to give, after crystallization from diisopropyl ether, 0.30 g (48%) of the title product as white crystals.

¹H-NMR (200 MHz, d₆-DMSO): δ [ppm]=0.86 (t, 3H), 1.15-1.61 (m, 4H), 2.46 (s, 3H), 2.84 (s, 3H), 3.01 (s, 3H), 3.30-3.47 (m, 1H), 3.52-3.72 (m, 4H), 3.74-3.95 (m, 1H), 4.36-4.59 (m, 2H), 4.69 (d, 1H), 5.53 (s, 1H), 6.56 (s, 1H), 7.21-7.48 (m, 5H).

### Commercial Utility

The compounds of the formulae 1, 1a and 2 and their pharmacologically acceptable salts (=active compounds according to the invention) have valuable pharmacological properties which make them commercially utilizable. In particular, they exhibit marked inhibition of gastric acid secretion and an excellent gastric and intestinal protective action in warm-blooded animals, in particular humans. In this connection, the active compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, peptic ulcer, including peptic ulcer bleeding, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics, such as NSAIDs and COX-inhibitors), chemicals (e.g. ethanol), gastric acid or stress situations. “Gastric and intestinal protection” is understood to include, according to general knowledge, gastroesophageal reflux disease (GERD), the symptoms of which include, but are not limited to, heartburn and/or acid regurgitation.

In their excellent properties, the active compounds according to the invention surprisingly prove to be clearly superior to the compounds known from the prior art in various models in which the antiulcerogenic and the antisecretory properties are determined. On account of these properties, the active compounds according to the invention are outstandingly suitable for use in human and veterinary medicine, where they are used, in particular, for the treatment and/or prophylaxis of disorders of the stomach and/or intestine.

A further subject of the invention are therefore the active compounds according to the invention for use in the treatment and/or prophylaxis of the abovementioned diseases.

The invention likewise includes the use of the active compounds according to the invention for the production of medicaments which are employed for the treatment and/or prophylaxis of the above-mentioned diseases.

The invention furthermore includes the use of the active compounds according to the invention for the treatment and/or prophylaxis of the abovementioned diseases.

A further subject of the invention are medicaments which comprise one or more active compounds according to the invention.

The medicaments are prepared by processes which are known per se and familiar to the person skilled in the art. As medicaments, the active compounds according to the invention (=active compounds) are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds can be administered orally, parenterally or percutaneously.

In general, it has proven advantageous in human medicine to administer the active compound(s) in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the active compounds according to the invention and/or their salts are to be used for the treatment of the abovementioned diseases, the pharmaceutical preparations can also contain one or more pharmacologically active constituents of other groups of medicaments, for example: tranquillizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino acids.

To be emphasized in this connection is in particular the combination of the active compounds according to the invention with pharmaceuticals which inhibit acid secretion, such as, for example, H₂ blockers (e.g. cimetidine, ranitidine), H⁺/K⁺ ATPase inhibitors (e.g. omeprazole, pantoprazole), or further with so-called peripheral anticholinergics (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, mezlocillin, ampicillin, amoxicillin, cefalothin, cefoxitin, cefotaxime, imipenem, gentamycin, amikacin, erythromycin, ciprofloxacin, metronidazole, clarithromycin, azithromycin and combinations thereof (for example clarithromycin+metronidazole).

In view of their excellent gastric and intestinal protection action, the active compounds according to the invention are suited for a free or fixed combination with those medicaments (e.g. certain antiinflammatories and antirheumatics, such as NSAIDs), which are known to have a certain ulcerogenic potency. In addition, the compounds of formula 1 are suited for a free or fixed combination with motility-modifying drugs.

### Pharmacology

The excellent gastric protective action and the gastric acid secretion-inhibiting action of the compounds according to the invention can be demonstrated in investigations on animal experimental models. The compounds according to the invention investigated in the model mentioned below have been provided with numbers which correspond to the numbers of these compounds in the examples.

### Testing of the Secretion-Inhibiting Action on the Perfused Rat Stomach

In Table A which follows, the influence of the compounds of the formula 1 according to the invention on the pentagastrin-stimulated acid secretion of the perfused rat stomach after intraduodenal administration in vivo is shown.

TABLE A

| | Dose | Inhibition of |
|---|---|---|
| | (μmol/kg) | acid secretion |
| No. | i.d. | (%) |
| 2 | 3.0 | >50 |

### Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion. The body temperature of the animals was kept at a constant 37.8-38° C. by infrared irradiation and heat pads (automatic, stepless control by means of a rectal temperature sensor).

1. A compound of the formula 1 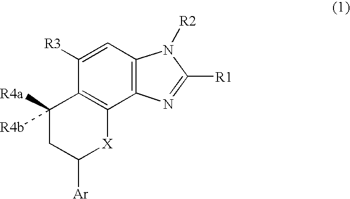
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl, hydroxy-1-4C-alkyl, mono- or di-1-4C-alkylamino or 1-4C-alkylcarbonyloxy-1-4C-alkyl,
R2 is hydrogen, 1-4C-alkyl, aryl, 3-7C-cycloalkyl, 3-7C-cycloalkyl-1-4C-alkyl, 1-4C-alkoxycarbonyl, mono- or di-1-4C-alkylamino 1-4C-alkylcarbonyl, hydroxy-1-4C-alkyl, fluoro-2-4C-alkyl, aryl-1-4C-alkoxy-1-4C-alkyl, hydroxy or 1-4C-alkoxy,
R3 is hydrogen, halogen, fluoro-1-4C-alkyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, 1-4C-alkoxy-1-4C-alkoxy, 1-4C-alkylcarbonylamino, 1-4C-alkylcarbonyl-N-1-4C-alkylamino, 1-4C-alkoxy-1-4C-alkylcarbonylamino or the group —CO—NR31R32,
where
R31 is hydrogen, hydroxy, 1-7C-alkyl, 3-7C-cycloalkyl, hydroxy-1-4C-alkyl or 1-4C-alkoxy-1-4C-alkyl and
R32 is hydrogen, 1-7C-alkyl, 3-7C-cycloalkyl, hydroxy-1-4C-alkyl or 1-4C-alkoxy-1-4C-alkyl,
or where
R31 and R32 together, including the nitrogen atom to which both are bonded, are a pyrrolidino, hydroxypyrrolidino, aziridino, azetidino, piperidino, piperazino, N-1-4C-alkylpiperazino or morpholino group,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl or 1-4C-alkoxy-1-4C-alkyl,
X is O (oxygen) or NH and
Ar is a mono- or bicyclic aromatic residue, substituted by R5, R6, R7 and R8, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
wherein
R5 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R6 is hydrogen, 1-4C-alkyl, 1-4C-alkoxy, 1-4C-alkoxycarbonyl, halogen, trifluoromethyl or hydroxy,
R7 is hydrogen, 1-4C-alkyl or halogen and
R8 is hydrogen, 1-4C-alkyl or halogen,
and wherein
aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano,
or a salt thereof.

2. A compound of formula 1 according to claim 1,
in which
R1 is hydrogen, 1-4C-alkyl, 3-7C-cycloalkyl or fluoro-1-4C-alkyl,
R2 is hydrogen or 1-4C-alkyl,
R3 is hydrogen, halogen, carboxyl; 1-4C-alkoxycarbonyl, hydroxy-1-4C-alkyl, 1-4C-alkoxy-1-4C-alkyl, 1-4C-alkoxy-1-4C-alkoxy-1-4C-alkyl or the group —CO—NR31R32,
where
R31 is hydrogen, 1-4C-alkyl, 3-7C-cycloalkyl, hydroxy-1-4C-alkyl or 1-4C-alkoxy-1-4C-alkyl and
R32 is hydrogen or 1-4C-alkyl,
or where
R31 and R32 together, including the nitrogen atom to which both are bonded, are a pyrrolidino, hydroxypyrrolidino, aziridino, azetidino, piperidino, piperazino, N-1-4C-alkylpiperazino or morpholino group,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl or 1-4C-alkoxy-1-4C-alkyl,
X is Q (oxygen) or NH and
Ar is a phenyl group, substituted by R5, R6, R7 and R8,
wherein
R5 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R6 is hydrogen, 1-4C-alkyl, 1-4C-alkoxy, 1-4C-alkoxycarbonyl, halogen, trifluoromethyl or hydroxy,
R7 is hydrogen, 1-4C-alkyl or halogen and
R8 is hydrogen, 1-4C-alkyl or halogen,
or a salt thereof.

3. A compound according to claim 1, characterized by the formula 1a, 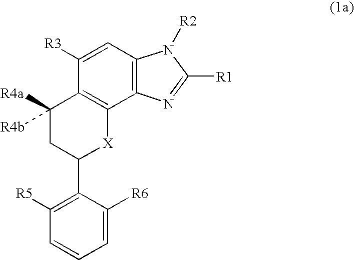
in which
R1 is hydrogen, 1-4C-alkyl, 3-7C-cycloalkyl or fluoro-1-4C-alkyl,
R2 is hydrogen or 1-4C-alkyl,
R3 is hydrogen, halogen, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl or the group —CO—NR31R32,
where
R31 is hydrogen, 1-4C-alkyl, 3-7C-cycloalkyl, hydroxy-1-4C-alkyl or 1-4C-alkoxy-1-4C-alkyl and
R32 is hydrogen or 1-4C-alkyl,
or where
R31 and R32 together, including the nitrogen atom to which both are bonded, are a pyrrolidino, hydroxypyrrolidino, aziridino, azetidino, piperidino, piperazino, N-1-4C-alkylpiperazino or morpholino group,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl,
R5 is hydrogen, 1-4C-alkyl hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino,
R6 is hydrogen, 1-4C-alkyl or 1-4C-alkoxy and
X is O (oxygen) or NH,
or a salt thereof.

4. A compound of formula 1a according to claim 3,
in which
R1 is 1-4C-alkyl, 3-7C-cycloalkyl or fluoro-1-4C-alkyl,
R2 is hydrogen or 1-4C-alkyl,
R3 is hydrogen, carboxyl, hydroxy-1-4C-alkyl, 1-4C-alkoxy-1-4C-alkyl, 1-4C-alkoxy-1-4C-alkoxy-1-4C-alkyl or the group —CO—NR31R32,
where
R31 is hydrogen, 1-4C-alkyl, hydroxy-1-4C-alkyl or 1-4C-alkoxy-1-4C-alkyl and
R32 is hydrogen or 1-4C-alkyl,
or where
R31 and R32 together, including the nitrogen atom to which both are bonded, are a pyrrolidino, aziridino, azetidino or morpholino group,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl,
R5 is hydrogen,
R6 is hydrogen and
X is O (oxygen) or NH,
or a salt thereof.

5. A compound of formula 1a according to claim 3,
in which
R1 is 1-4C-alkyl,
R2 is hydrogen or 1-4C-alkyl,
R3 is hydrogen or the group —CO—NR31R32,
where
R31 is hydrogen or 1-4C-alkyl and
R32 is 1-4C-alkyl,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl,
R5 is hydrogen,
R6 is hydrogen and
X is O (oxygen) or NH,
or a salt thereof.

6. A compound of formula 1a according to claim 3,
in which
R1 is 1-4C-alkyl,
R2 is 1-4C-alkyl,
R3 is hydrogen or the group —CO—NR31R32,
where
R31 is 1-4C-alkyl and
R32 is 1-4C-alkyl,
one of R4a and R4b is hydrogen and the other is hydroxy, 1-4C-alkoxy or 1-4C-alkoxy-1-4C-alkoxy,
R5 is hydrogen,
R6 is hydrogen and
X is O (oxygen) or NH,
or a salt thereof.

7. A compound according to claim 1, characterized by the formula 2, 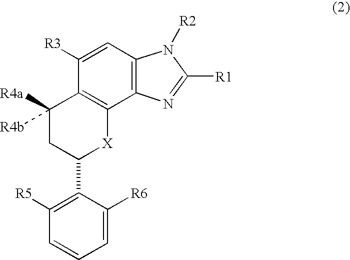
in which
R1 is 1-4C-alkyl or 3-7C-cycloalkyl,
R2 is hydrogen or 1-4C-alkyl,
R3 is 1-4C-alkoxycarbonyl, hydroxy-1-4C-alkyl, 1-4C-alkoxy-1-4C-alkyl or the group —CO—NR31R32,
where
R31 is hydrogen, 1-4C-alkyl, hydroxy-1-4C-alkyl or 1-4C-alkoxy-1-4C-alkyl and
R32 is hydrogen or 1-4C-alkyl,
or where
R31 and R32 together, including the nitrogen atom to which both are bonded, are a pyrrolidino, aziridino, azetidino or morpholino group,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl or 1-4C-alkoxy-1-4C-alkyl,
R5 is hydrogen,
R6 is hydrogen and
X is O (oxygen) or NH,
or a salt thereof.

8. A compound of formula 2 according to claim 7,
in which
R1 is 1-4C-alkyl,
R2 is hydrogen or 1-4C-alkyl,
R3 is hydrogen or the group —CO—NR31R32,
where
R31 is hydrogen or 1-4.C-alkyl and
R32 is 1-4C-alkyl,
one of R4a and R4b is hydrogen and the other is hydroxy, 1-4C-alkoxy or 1-4C-alkoxy-1-4C-alkoxy, or one of R4a and R4b is hydroxy and the other is 1-4C-alkyl,
R5 is hydrogen,
R6 is hydrogen and
X is O (oxygen) or NH,
or a salt thereof.

9. A compound of formula 2 according to claim 7,
in which
R1 is 1-4C-alkyl,
R2 is 1-4C-alkyl,
R3 is hydrogen or the group —CO—NR31R32,
where
R31 is 1-4C-alkyl and
R32 is 1-4C-alkyl,
one of R4a and R4b is hydrogen, and the other is hydroxy, 1-4C-alkoxy or 1-4C-alkoxy-1-4C-alkoxy,
R5 is hydrogen,
R6 is hydrogen and
X is O (oxygen) or NH,
or a salt thereof.

10. A pharmaceutical composition comprising a compound as claimed in claim 1 and/or a pharmacologically acceptable salt thereof together with a pharmaceutically acceptable auxiliary and/or excipient.

11. (canceled)

12. A method of preventing or treating a gastrointestinal disorder in a patient comprising administering to a patient in need thereof a therapeutically effective amount of a compound as claimed in claim 1, or a pharmacologically acceptable salt thereof.
